(19) United States
(12) Patent Application Publication
(10) Pub. No.: US 2006/0089347 A1
(43) Pub. Date: Apr. 27, 2006

(54) Substituted azepines as histamine h3 receptor antagonists, preparation and therapeutic uses

(75) Inventors: Robert Alan Gadski, Indianapolis, IN (US); Phillip Arthur Hipskind, New Palestine, IN (US); Cynthia Darshini Jesudason, Indianapolis, IN (US); Richard Todd Pickard, Noblesville, IN (US); Lisa Selsam Beavers, Franklin, IN (US); Christopher Stephen Sledem, Longmont, CO (US); Alay Singh, Aurora, CA (US)

Correspondence Address: ELI LILLY & COMPANY, PATENT DIVISION, P.O. BOX 6288, INDIANAPOLIS, IN, 46206-6288, US

(73) Assignee: Eli Lilly and Company, Indianapolis, IN (US)

(21) Appl. No.: 10/523,071

(22) PCT Filed: Aug. 15, 2003
(86) PCT No.: PCT/US03/23266

§ 371 Date: Oct. 18, 2005

Related U.S. Application Data
(60) Provisional application No. 60/405,053, filed on Aug. 20, 2002.

(51) Int. Cl. A61K 31/55; C07D 223/16

(52) U.S. Cl. 514/212.07; 514/217.01; 540/523; 540/593

(57) ABSTRACT

The present invention discloses novel substituted azepine compounds of Formula (I) or pharmaceutically acceptable salts thereof which have selective histamine-H3 receptor antagonist activity as well as methods for preparing such compounds. In another embodiment, the invention discloses pharmaceutical compositions comprising such azepines as well as methods of using them to treat obesity and other histamine H3 receptor-related diseases.

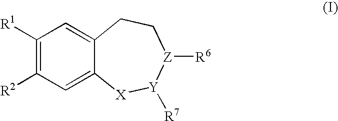

The present invention relates to histamine H3 receptor antagonists, and as such are useful in the treatment of disorders responsive to the inactivation of histamine H3 receptors, such as obesity, cognitive disorders, attention deficit disorders and the like.

The histamine H3 receptor (H3R) is a presynaptic autoreceptor and hetero-receptor found in the peripheral and central nervous system and regulates the release of histamine and other neurotransmitters, such as serotonin and acetylcholine. The histamine H3 receptor is relatively neuron specific and inhibits the release of a number of monoamines, including histamine. Selective antagonism of the histamine H3 receptor raises brain histamine levels and inhibits such activities as food consumption while minimizing non-specific peripheral consequences. Antagonists of the histamine H3 receptor increase synthesis and release of cerebral histamine and other monoamines. By this mechanism, they induce a prolonged wakefulness, improved cognitive function, reduction in food intake and normalization of vestibular reflexes. Accordingly, the histamine H3 receptor is an important target for new therapeutics in Alzheimer disease, mood and attention adjustments, cognitive deficiencies, obesity, dizziness, schizophrenia, epilepsy, sleeping disorders, narcolepsy and motion sickness.

The majority of histamine H3 receptor antagonists to date resemble histamine in possessing an imidazole ring generally substituted in the 4(5) position (Ganellin et al., Ars Pharmaceutica, 1995, 36:3, 455-468). A variety of patents and patent applications directed to antagonists and agonists having such structures include EP 197840, EP 494010, WO 97/29092, WO 96138141, and W096/38142. These imidazole-containing compounds have the disadvantage of poor blood-brain barrier penetration, interaction with cytochrome P-450 proteins, and hepatic and ocular toxicities.

Non-imidazole neuroactive compounds such as beta histamines (Arrang, Eur. J. Pharm. 1985, 111:72-84) demonstrated some histamine H3 receptor activity but with poor potency. EP 978512 published Mar. 1, 2000 discloses non-imidazole aryloxy alkylamines as histamine H3 receptor antagonists, but does not disclose the affinity, if any, of these antagonists for recently identified histamine receptor GPRv53, described below. EP 0982300A2 (pub. Mar. 1, 2000) discloses non-imidazole alkylamines as histamine HS receptor ligand which are similar to the subject invention by having a phenoxy core structure although the subject invention is unique in the presence of a saturated, fused heterocyclic ring appended to the central benzene core. Furthermore the compounds of this invention are highly selective for the H3 receptor (vs. other histamine receptors), and possess advantageous drug disposition properties (pharmacolinetics).

Histamine mediates its activity via four receptor subtypes, H1R, H2R, H3R and a newly identified receptor designated GPRv53 [(Oda T., et al., J. Biol. Chem. 275 (47): 36781-6 (2000)]. Alternative names for the GPRv53 receptor are PORT3 or H4R. Although relatively selective ligands have been developed for H1R, H2R and H3R, few specific ligands have been developed that can distinguish H3R from GPRv53. GPRv53 is a widely distributed receptor found at high levels in human leukocytes. Activation or inhibition of this receptor could result in undesirable side effects when targeting antagonism of the H3R receptor. Furthermore, the identification of this new receptor has fundamentally changed histamine biology and must be considered in the development of histamine H3 receptor antagonists.

Because of the unresolved deficiencies of the compounds described above, there is a continuing need for improved methods and compositions to treat disorders associated with histamine H3 receptors. The present invention provides compounds that are useful as histamine H3 receptor antagonists. In another aspect, the present invention provides compounds that are useful as selective antagonists of the histamine H3 receptor but have little or no binding affinity of GPRv53. In yet another aspect, the present invention provides pharmaceutical compositions comprising antagonists of the histamine H3 receptor. In yet another aspect, the present invention provides compounds, pharmaceutical compositions, and methods useful in the treatment of obesity, cognitive disorders, attention deficit disorders and other disorders associated with histamine H3 receptor.

The present invention is a compound structurally represented by Formula I 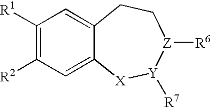
or pharmaceutically acceptable salts thereof, wherein:
R¹ and R² are independently H, or —OR³N R⁴R⁵, provided only one of R¹ and R² can be —OR³NR⁴R⁵;
R³ is (C₂-C₅)alkylene;
R⁴ is (C₁-C₄)alkyl;
R⁵ is (C₁-C₄)alkyl,
   wherein R⁴ and R⁵ taken together with the nitrogen atom to which they are attached can form a piperidinyl or pyrrolidinyl ring;
X is CH₂ or CO;
Y and Z are —CH₂— or N, provided only one of Y and Z can be N;
R⁶ is hydrogen,
   —(C₁-C₄)alkyl,
   —CH₂-phenyl,
   —CH₂(C₃-C₇)cycloalkyl,
   —CO₂R⁸,
   —SO₂R⁹,
   —CONHR¹⁰,
   —COR¹¹,
   —CH₂CH₂NR¹²R¹³, or
   —CH₂R¹⁴;
R⁷ is hydrogen,
   —(C₁-C₄)alkyl,
   —CH₂-phenyl,
   —CH₂(C₃-C₇)cycloalkyl,
   —CO₂R⁸,
   —SO₂R⁹,
   —CONHR¹⁰,
   —COR¹¹,
   —CH₂CH₂NR¹²R¹³, or
   —CH₂R¹⁴;
Wherein;
R⁸ is
   —(C₁-C₄)alkyl, or
   —(C₃-C₇)cycloalkyl;
R⁹ is
   —(C₁-C₄)alkyl,
   —(C₃-C₇)cycloalkyl, or
   -phenyl;
R¹⁰ is
   —(C₁-C₄)alkyl, or
   —(C₃-C₇)cycloalkyl;
R¹¹ is
   —(C₁-C₄)alkyl,
   —(C₃-C₇)cycloalkyl,
   —CH₂NR¹²R¹³, or
   —(C₃-C₇)cycloalkyl, wherein optionally one or more of said carbons is replaced by N, NR¹⁰, or NCO₂R¹⁰;
R¹² is
   -hydrogen, or
   —(C₁-C₄)alkyl;
R¹³ is
   -hydrogen,
   —(C₁-C₄)alkyl,
   —CO₂R¹⁰, or
   -phenyl;
R¹⁴ is
   —(C₁-C₄)alkyl, or
   (C₃-C₇)cycloalkyl, wherein optionally one or more of said carbons is replaced by N, NR¹⁰, or NCO₂R¹⁰.

While all of the compounds of the present invention are useful, certain of the compounds are particularly interesting and are preferred. The following listing sets out several groups of preferred compounds. It will be understood that each of the listings may be combined with other listings to create additional groups of preferred embodiments.
1) R¹ is —OR³NR⁴R⁵
2) R² is hydrogen
3) R³ is —CH₂CH₂CH₂—
4) R⁴ and R⁵ cyclize with the nitrogen to which they are attached to form a piperidinyl ring
5) Y is N
6) Z is CH₂

Alternatively, R² is —R³NR⁴R⁵, R¹ is hydrogen, R³ is —CH₂CH₂CH₂—, R⁴ and R⁵ cyclize with the nitrogen to which they are attached to form a piperidinyl ring, Y is N and Z is CH₂. Alternatively, R² is —OR³NR⁴R⁵, R¹ is hydrogen, R³ is —CH₂CH₂CH₂—, R⁴ and R⁵ cyclize with the nitrogen to which they are attached to form a piperidinyl ring, Z is N and Y is CH₂.

The present invention is a pharmaceutical composition which comprises a compound of Formula I and a pharmaceutically acceptable carrier. Pharmaceutical formulations of Formula I can provide a method of selectively increasing histamine levels in cells by contacting the cells with an antagonist of the histamine H3 receptor, the antagonists being a compound of Formula I.

The present invention further provides an antagonist of Formula I which is characterized by having little or no binding affinity for the histamine receptor GPRv53. Thus, a pharmaceutical preparation of Formula I can be useful in the treatment or prevention of obesity, cognitive disorders, attention deficit disorders and the like, which comprises administering to a subject in need of such treatment or prevention an effective amount of a compound of Formula I. In addition, a pharmaceutical preparation of Formula I can be useful in the treatment or prevention of a disorder or disease in which inhibition of the histamine H3 receptor has a beneficial effect or the treatment or prevention of eating disorders which comprises administering to a subject in need of such treatment or prevention an effective amount of a compound of Formula I.

General terms used in the description of compounds, compositions, and methods herein described, bear their usual meanings. Throughout the instant application, the following terms have the indicated meanings:

The term “GPRv53” means a recently identified novel histamine receptor as described in Oda, et al., supra. Alternative names for this receptor are PORT3 or H4R.

The term “H3R” means to the histamine H3 receptor that inhibits the release of a number of monoamines, including histamine.

The term “H1R” means to the histamine H1 receptor subtype.

The term “H2R” means to the histamine H2 receptor subtype.

The term “selective H3R antagonists” is defined as the ability of a compound of the present invention to block forskolin-stimulated cAMP production in response to agonist R(−)α methylhistamine.

In the general formulae of the present document, the general chemical terms have their usual meanings. For example:

“Alkylene” are a saturated hydrocarbyldiyl radical of straight or branched configuration made up of from 2 to 5 carbon atoms. Included within the scope of this term are ethylene, propylene, and the like.

“Alkyl” are one to four or one to eight carbon atoms such as methyl, ethyl, propyl, butyl, pentyl, hexyl, heptyl, octyl and isomeric forms thereof.

“Boc” or “BOC” refer to t-butyl carbamate.

“HOBT” is 1-hydrobenzotriazole.

“Cycloalkyl” are three to seven carbon atoms such as cyclopropyl, cyclobutyl, cyclopentyl and cyclohexyl.

“Halogen” or “halo” means fluoro, chloro, bromo and iodo.

“PS-Trisamine” is Tris-(2-aminoethyl)amine polystyrene. “PS-Carbodiimide” or “PS-CDI” is N-Cyclohexylcarbodiimide-N′-propyloxymethyl polystyrene. “PS-DIEA” is N,N-(Diisopropyl)aminomethylpolystyrene (1% inorganic antistatic agent). “PS-DMAP” is N-(methylpolystyrene)-4-(methylamino)pyridine.

“Composition” means a pharmaceutical composition and is intended to encompass a pharmaceutical product comprising the active ingredient(s), Formula I, and the inert ingredient(s) that make up the carrier. Accordingly, the pharmaceutical compositions of the present invention encompass any composition made by admixing a compound of the present invention and a pharmaceutically acceptable carrier.

The term “unit dosage form” means physically discrete units suitable as unitary dosages for human subjects and other non-human animals, each unit containing a predetermined quantity of active material calculated to produce the desired therapeutic effect, in association with a suitable pharmaceutical carrier.

The terms “treat”, “treating”, and “treatment”, as used herein, include their generally accepted meanings, i.e., preventing, prohibiting, restraining, alleviating, ameliorating, slowing, stopping, or reversing the progression or severity of a pathological condition, described herein.

While all of the compounds of the present invention are useful, certain of the compounds are particularly interesting and are preferred. The following listing sets out several groups of preferred compounds. It will be understood that each of the listings may be combined with other listings to create additional groups of preferred embodiments.
1) R¹ is —OR³NR⁴R⁵
2) R² is hydrogen
3) R³ is —CH₂CH₂CH₂—
4) R⁴ and R⁵ cyclize with the nitrogen to which they are attached to form a piperidinyl ring
5) Y is N
6) Z is CH₂

Alternatively, R² is —OR³NR⁴R⁵, R¹ is hydrogen, R³ is —CH₂CH₂CH₂—, R⁴ and R⁵ cyclize with the nitrogen to which they are attached to form a piperidinyl ring, Y is N and Z is CH₂. Alternatively, R² is —R³NR⁴R⁵, R¹ is hydrogen, R³ is —CH₂CH₂CH₂—, R⁴ and R⁵ cyclize with the nitrogen to which they are attached to form a piperidinyl ring, Z is N and Y is CH₂.

It will be understood that, as used herein, references to the compounds of Formula I are meant to also include the pharmaceutical salts, tautomers, enantiomers and other stereoisomers of the compounds, and racemic mixtures thereof. Thus, as one skilled in the art knows, certain aryls may exist in tautomeric forms. Such variations are contemplated to be within the scope of the invention.

Some of the compounds of the present invention have one or more chiral centers and may exist in a variety of stereoisomeric configurations. As a consequence of these chiral centers, the compounds of the present invention occur as racemates, mixtures of enantiomers and as individual enantiomers, as well as diastereomers and mixtures of diastereomers. All such racemates, enantiomers, and diastereomers are within the scope of the present invention.

As used herein, the term “stereoisomer” refers to a compound made up of the same atoms bonded by the same bonds but having different three-dimensional structures which are not interchangeable. The three-dimensional structures are called configurations. As used herein, the term “enantiomer” refers to two stereoisomers whose molecules are nonsuperimposable mirror images of one another. The term “chiral center” refers to a carbon atom to which four different groups are attached. As used herein, the term “diastereomers” refers to stereoisomers which are not enantiomers. In addition, two diastereomers which have a different configuration at only one chiral center are referred to herein as “epimers”. The terms “racemate”, “racemic mixture” or “racemic modification” refer to a mixture of equal parts of enantiomers.

The term “enantiomeric enrichment” as used herein refers to the increase in the amount of one enantiomer as compared to the other. A convenient method of expressing the enantiomeric enrichment achieved is the concept of enantiomeric excess, or “ee”, which is found using the following equation:

$$ee = \frac{{E}^{1} - {E}^{2}}{{E}^{1} + {E}^{2}} \times 100$$

wherein E¹ is the amount of the first enantiomer and E² is the amount of the second enantiomer. Thus, if the initial ratio of the two enantiomers is 50:50, such as is present in a racemic mixture, and an enantiomeric enrichment sufficient to produce a final ratio of 70:30 is achieved, the ee with respect to the first enantiomer is 40%. However, if the final ratio is 90:10, the ee with respect to the first enantiomer is 80%. An ee of greater than 90% is preferred, an ee of greater than 95% is most preferred and an ee of greater than 99% is most especially preferred. Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art. In addition, the specific stereoisomers and enantiomers of compounds of formula I can be prepared by one of ordinary skill in the art utilizing well known techniques and processes, such as those disclosed by J. Jacques, et al., “Enantiomers, Racemates, and Resolutions”, John Wiley and Sons, Inc., 1981, and E. L. Eliel and S. H. Wilen, “Stereochemistry of Organic Compounds”, (Wiley-Interscience 1994), and European Patent Application No. EP-A-838448, published Apr. 29, 1998. Examples of resolutions include recrystallization techniques or chiral chromatography.

The compounds of Formula I, when existing as a diastereomeric mixture, may be separated into diastereomeric pairs of enantiomers by, for example, fractional crystallization from a suitable solvent, for example methanol or ethyl acetate or a mixture thereof. The pair of enantiomers thus obtained may be separated into individual stereoisomers by conventional means, for example by the use of an optically active acid as a resolving agent. Alternatively, any enantiomer of a compound of the formula may be obtained by stereospecific synthesis using optically pure starting materials or reagents of known configuration or through enantioselective synthesis.

The terms “R” and “S” are used herein as commonly used in organic chemistry to denote specific configuration of a chiral center. The term “R” (rectus) refers to that configuration of a chiral center with a clockwise relationship of group priorities (highest to second lowest) when viewed along the bond toward the lowest priority group. The term “S” (sinister) refers to that configuration of a chiral center with a counterclockwise relationship of group priorities (highest to second lowest) when viewed along the bond toward the lowest priority group. The priority of groups is based upon their atomic number (in order of decreasing atomic number). A partial list of priorities and a discussion of stereochemistry is contained in “Nomenclature of Organic Compounds: Principles and Practice”, (J. H. Fletcher, et al., eds., 1974) at pages 103-120.

The designation “ [symbol] ” refers to a bond that protrudes forward out of the plane of the page. The designation “ [symbol] ” refers to a bond that protrudes backward out of the plane of the page. The designation “ [symbol] ” refers to a bond wherein the stereochemistry is not defined.

In general, the term “pharmaceutical” when used as an adjective means substantially non-toxic to living organisms. For example, the term “pharmaceutical salt” as used herein, refers to salts of the compounds of formula I which are substantially non-toxic to living organisms. See, e.g., Berge, S. M, Bighley, L. D., and Monkhouse, D. C., “Pharmaceutical Salts” J. Pharm. Sci., 66:1, 1977. Typical pharmaceutical salts include those salts prepared by reaction of the compounds of formula I with an inorganic or organic acid or base. Such salts are known as acid addition or base addition salts respectively. These pharmaceutical salts frequently have enhanced solubility characteristics compared to the compound from which they are derived, and thus are often more amenable to formulation as liquids or emulsions.

The term “acid addition salt” refers to a salt of a compound of formula I prepared by reaction of a compound of formula I with a mineral or organic acid. For exemplification of pharmaceutical acid addition salts see, e.g., Berge, S. M, Bighley, L. D., and Monkhouse, D. C., J. Pharm. Sci., 66:1, 1977. Since compounds of this invention can be basic in nature, they accordingly react with any of a number of inorganic and organic acids to form pharmaceutical acid addition salts.

Such acid addition salts include sulfate, pyrosulfate, bisulfate, sulfite, bisulfite, phosphate, mono-hydrogenphosphate, dihydrogenphosphate, metaphosphate, pyrophosphate, chloride, bromide, iodide, acetate, propionate, decanoate, caprylate, acrylate, formate, isobutyrate, heptanoate, propiolate, oxalate, malonate, succinate, suberate, sebacate, fumarate, maleate, 2-butyne-1,4 dioate, 3-hexyne-2,5-dioate, benzoate, chlorobenzoate, hydroxybenzoate, methoxybenzoate, phthalate, xylenesulfonate, phenylacetate, phenylpropionate, phenylbutyrate, citrate, lactate, hippurate, beta-hydroxybutyrate, glycollate, maleate, tartrate, methanesulfonate, propanesulfonate, naphthalene-1-sulfonate, naphthalene-2-sulfonate, mandelate and the like salts.

The pharmaceutical acid addition salts of the invention are typically formed by reacting the compound of formula I with an equimolar or excess amount of acid. The reactants are generally combined in a mutual solvent such as diethylether, tetrahydrofuran, methanol, ethanol, isopropanol, benzene, and the like. The salts normally precipitate out of solution within about one hour to about ten days and can be isolated by filtration or other conventional methods.

Acids commonly employed to form acid addition salts are inorganic acids such as hydrochloric acid, hydrobromic acid, hydroiodic acid, sulfuric acid, phosphoric acid, and the like, and acids commonly employed to form such salts are inorganic acids such as hydrochloric acid, hydrobromic acid, hydroiodic acid, sulfuric acid, phosphoric acid, and the like, and organic acids, such as p-toluenesulfonic acid, methanesulfonic acid, oxalic acid, p-bromophenylsulfonic acid, carbonic acid, succinic acid, citric acid, benzoic acid, acetic acid and the like. Examples of such pharmaceutically acceptable salts thus are the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, β-hydroxybutyrate, glycollate, tartrate, methanesulfonate, propanesulfonate, naphthalene-1-sulfonate, naphthalene-2-sulfonate, mandelate and the like.

The term “base addition salt” refers to a salt of a compound of formula I prepared by reaction of a compound of formula I with a mineral or organic base. For exemplification of pharmaceutical base addition salts see, e.g., Berge, S. M, Bighley, L. D., and Monkhouse, D. C., J. Pharm. Sci., 66:1, 1977. This invention also contemplates pharmaceutical base addition salts of compounds of formula I. The skilled artisan would appreciate that some compounds of formula I may be acidic in nature and accordingly react with any of a number of inorganic and organic bases to form pharmaceutical base addition salts. Examples of pharmaceutical base addition salts are the ammonium, lithium, potassium, sodium, calcium, magnesium, methylamino, diethylamino, ethylene diamino, cyclohexylamino, and ethanolamino salts, and the like of a compound of formula I.

It should be recognized that the particular counterion forming a part of any salt of this invention is not of a critical nature, so long as the salt as a whole is pharmacologically acceptable and as long as the counterion does not contribute undesired qualities to the salt as a whole.

The compounds of Formula I can be prepared by one of ordinary skill in the art following a variety of procedures, some of which are illustrated in the procedures and schemes set forth below. The particular order of steps required to produce the compounds of formula I is dependent upon the particular compound to being synthesized, the starting compound, and the relative liability of the substituted moieties. The reagents or starting materials are readily available to one of skill in the art, and to the extent not commercially available, are readily synthesized by one of ordinary skill in the art following standard procedures commonly employed in the art, along with the various procedures and schemes set forth below, including, for example, Schemes 1 and 2.

The following Preparations and Examples are provided to better elucidate the practice of the present invention and should not be interpreted in any way as to limit the scope of the same. Those skilled in the art will recognize that various modifications may be made while not departing from the spirit and scope of the invention. All publications mentioned in the specification are indicative of the level of those skilled in the art to which this invention pertains.

The terms and abbreviations used in the instant Preparations and Examples have their normal meanings unless otherwise designated. For example, as used herein, the following terms have the meanings indicated: “eq” refers to equivalents; “N” refers to normal or normality, “M” refers to molar or molarity, “g” refers to gram or grams, “mg” refers to milligrams; “L” refers to liters; “mL” refers to milliliters; “μL” refers to microliters; “mol” refers to moles; “mmol” refers to millimoles; “psi” refers to pounds per square inch; “min” refers to minutes; “h” or “hr” refers to hours; “° C.” refers to degrees Celsius; “TLC” refers to thin layer chromatography; “HPLC” refers to high performance liquid chromatography; “R_{f}” refers to retention factor; “Rₜ” refers to retention time; “δ” refers to part per million down-field from tetramethylsilane; “MS” refers to mass spectrometry, Observed Mass indicates (M+1) unless indicated otherwise. “UV” refers to ultraviolet spectrometry, “¹H NMR” refers to proton nuclear magnetic resonance spectrometry. In addition, “IR” refers to infrared spectrometry, and the absorption maxima listed for the IR spectra are only those of interest and not all of the maxima observed. “RT” refers to room temperature. 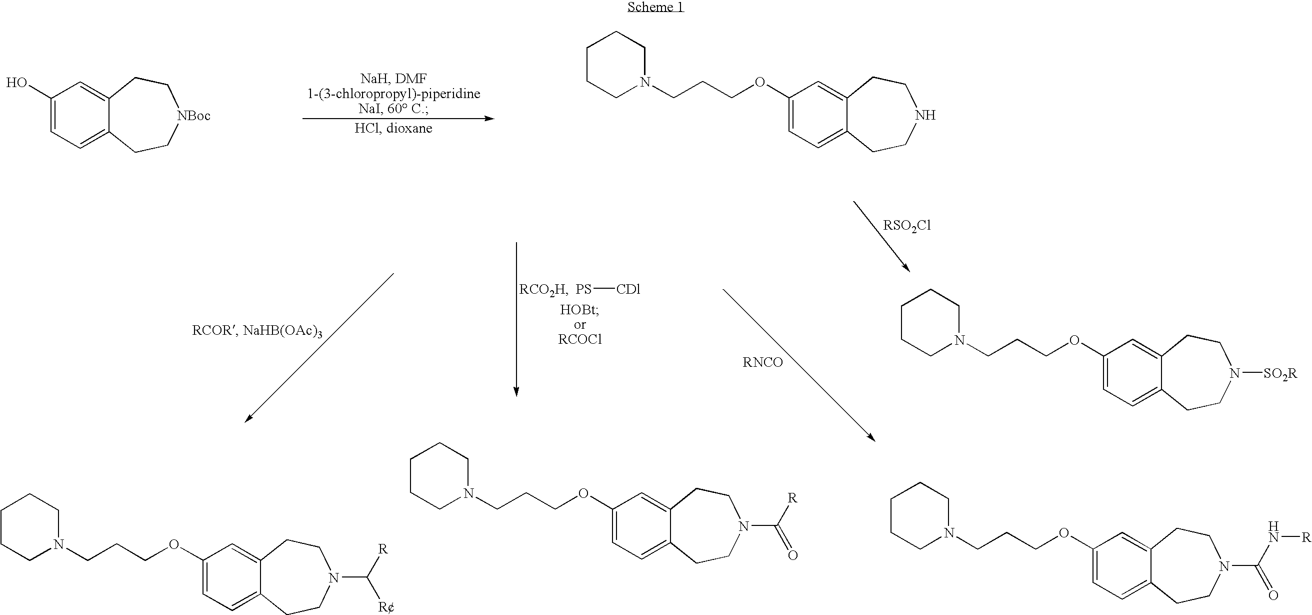 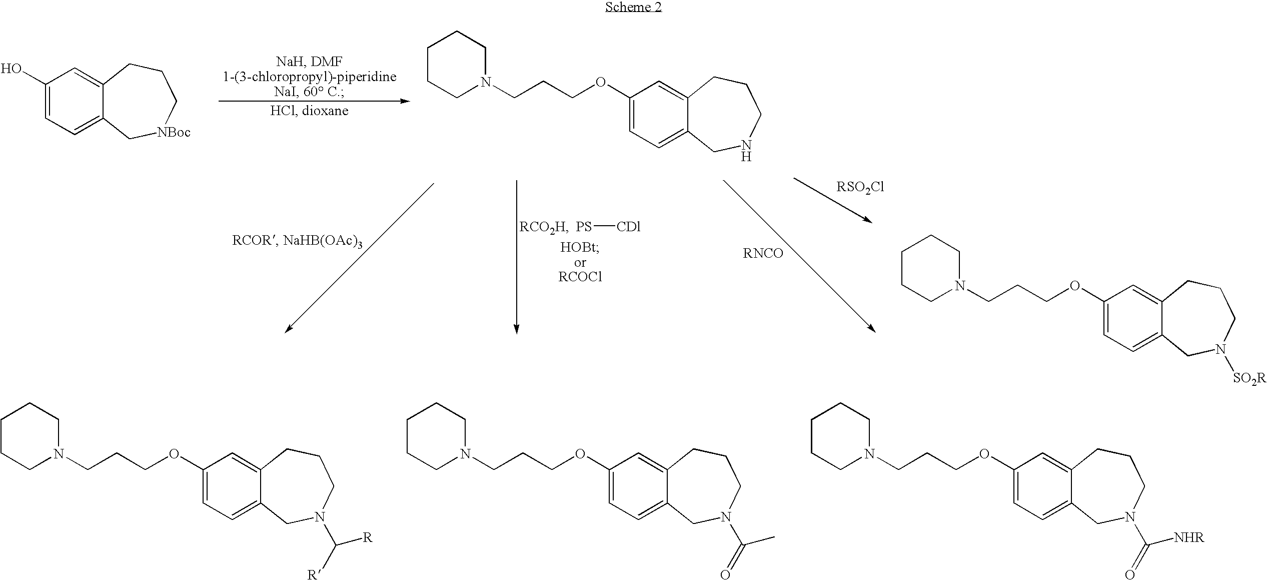 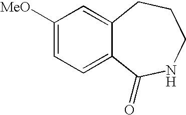
Preparation 1.

7-Methoxy-2,3,4,5-tetrahydro-benzo[c]azepin-1-one is prepared by the procedures similar to those described in Shtacher, G.; Erez, M.; Cohen, S. J Med Chem 1973, 16, 516. 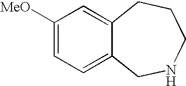
Preparation 2.

### 7-methoxy-2,3,4,5,5-tetrahydro-benzo[c]azepine

7-Methoxy-2,3,4,5-tetrahydro-benzo[c]azepin-1-one (10 g, 53 mmol) was added to THF (50 mL) under a nitrogen atmosphere. The stirring solution was chilled to 0° C. via ice bath and borane-THF complex (156 ml, 1M in THF, 156 mmol) was added dropwise. After complete addition, the solution was refluxed for 2 hours and cooled to room temperature. The solution was quenched with 1 M HCl solution. The pH was adjusted to 9 with 1N NaOH solution and 300 mL of EtOAc was added. The solution was extracted and the organic layer was dried over magnesium sulfate and concentrated to a yellow oil. The oil was chromatographed on a biotage 75s column (10% MeOH/DCM) to yield 4.2 grams of the title compound as a white solid (45% of theory) ¹H NMR (DMSO) δ7.00 (d, 1 H), 6.63 (s, 1H), 6.59 (dd, 1 H), 3.67 (s, 3 H), 3.02 (t, 2 H, 2.72 (m, 2 H), 1.55 (m, 2 H). MS (EI) 178.2 m/z (M+) 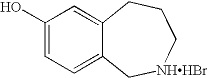
Preparation 3.

### 2,3,4,5-tetrahydro-1H-benzo[c]azepin-7-ol hydrobromide

7-methoxy-2,3,4,5,5-tetrahydro-benzo[c]azepine (4.2 g, 22 mmol) was dissolved in methylene chloride (50 mL) and added to boron tribromide (67 mmol, 6.4 mL) in methylene chloride (20 mL) at −78° C. under a nitrogen atmosphere. The temperature was maintained below −70° C. The reaction was stirred at −70° C. for 2 hours and the ice bath was removed. The reaction was stirred at room temperature for 16 hours. The clear solution was cooled to −78° C. and methanol (15 mL) was carefully added. The solution was then concentrated to a brown solid. The solid was dissolved in methanol (50 mL) and methylene chloride (40 mL) was added. The solution was concentrated to half-volume and hexanes were added (40 mL). The solution was concentrated to half volume and ethyl acetate (20 mL) was added. The solution was concentrated to a volume to 20 mL and the solution was filtered to obtain a white granular solid (4.2 g, 45% of theory) ¹H NMR (DMSO) δ 9.52 (s, 1H), 8.70 (br, 2H), 7.19 (d, 1H), 6.58 (m, 2H), 4.23 (s, 2H), 3.33 (m, 2H), 2.88 (m, 2H), 1.70 (m, 2H). MS (ES) 164.1 m/z (M-HBr). Elemental analysis; Calculated values: C, 49.19; H, 5.78; N, 5.55; Observed values: C, 49.48; H, 5.78; N, 5.55. 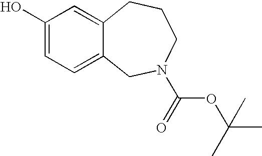
Preparation 4.

### 7-Hydroxy-1,3,4,5-tetrahydro-benzo[c]azepine-2-carboxylic acid tert-butyl ester

2,3,4,5-tetrahydro-1H-benzo[c]azepin-7-ol hydrobromide (6.50 g, 26 mmol) was slurried in methylene chloride (100 mL). Triethylamine (79 mmol) was added and the slurry was cooled to 5° C. via ice bath. BOC anhydride was dissolved in methylene chloride (20 mL) and added dropwise to the solution. The ice bath was removed and the solution was allowed to stir at room temperature for four hours. The solution was concentrated to a brown solid and 40 ml of a 1:1 methylene chloride/EtOAc solution was added and the solution was filtered. The filtrate was concentrated to a brown oil that was chromatographed (20% EtOAc/Hex) to give a white solid (6.3 g, 90% of theory). ¹H NMR (DMSO) δ9.15 (s, 1H), 6.97 (d, 1H), 6.60 (s, 1H), 6.49 (d, 1H), 4.23 (s, 2H), 3.52 (br m, 2H), 2.72 (br m, 2H), 1.59 (br m, 2H), 1.33 (s, 9). ¹³C NMR (DMSO) δ 156.24, 142.99, 129.41, 116.41, 111.57, 78.29, 50.95, 49.57, 34.58, 28.02. Elemental analysis; Calculated values: C, 68.42; H, 8.04; N, 5.32; Observed values: C, 68.54; H, 8.15; 5.24. 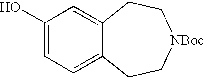
Preparation 5.

7-Hydroxy-1,2,4,5-tetrahydro-benzoldlazepine-3-carboxylic acid tert-butyl ester; prepared by the method reported in Austin et al., Bioorganic Med Chem Letts, 2000, 10, 2553. 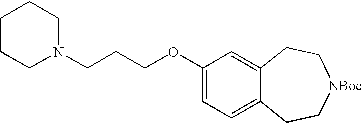
Preparation 6.

7-(3-Piperidin-1-yl-propoxy)-1,2,4,5-tetrahydro-benzo[d]azepine-3-carboxylic acid tert-butyl ester; prepared in quantitative yield from 7-hydroxy-1,2,4,5-tetrahydro-benzo[d]azepine-3-carboxylic acid tert-butyl ester (2 g, 7.6 mmol) and 1-(3-chloropropyl)-piperidine (1.5 mL, 9.3 mmol) by the method of Procedure A. (See herein example 1) A portion was purified by flash chromatography on silica gel (30:1 DCM/7N NH₃ in methanol). MS (ESI), M+H: 389 (100%).

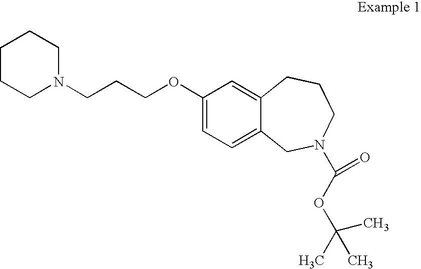

Procedure A: To a stirred solution of 7-hydroxy-1,3,4,5-tetrahydro-benzo[c]azepine-2-carboxylic acid tert-butyl ester (2 g, 7.6 mmol) in dry dimethylformamide (DMF) (16 mL) at room temperature under N₂, is added sodium hydride (60% dispersion, 0.36 g, 9.12 mmol) portion wise. The mixture is stirred for 15 minutes, and 1-(3-chloropropyl)-piperidine (1.5 mL, 9.3 mmol) is added, followed by sodium iodide (1.09 g, 7.23 mmol). After heating for 4 hours at 70° C., the reaction mixture is cooled to room temperature, poured into water, extracted three times with ethyl acetate, dried over anhydrous potassium carbonate and concentrated in vacuo, to provide quantitatively, 7-(3-Piperidin-1-yl-propoxy)-1,3,4,5-tetrahydro-benzo[c]azepine-2-carboxylic acid tert-butyl ester. A portion was purified by flash chromatography on silica gel (30:1 DCM/7N NH₃ in methanol). MS (ESI), M+H: 389 (100%).

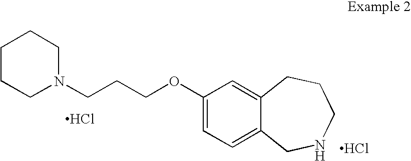

7-(3-Piperidin-1-yl-propoxy)-2,3,4,5-tetrahydro-1H-benzo[c]azepine dihydrochloride; prepared (2.6 g, 100%) from 7-(3-piperidin-1-yl-propoxy)-1,3,4,5-tetrahydro-benzo[c]azepine-2-carboxylic acid tert-butyl ester (2.8 g, 7.22 mmol) by the method of Procedure H. (See herein Example 22). MS (APCI), M+H: 289 (100%).

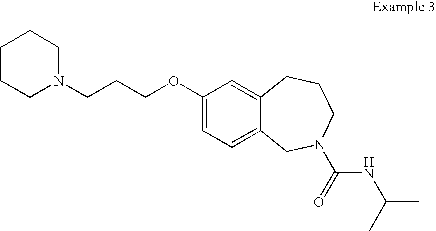

7-(3-Piperidin-1-yl-propoxy)-1,2,4,5-tetrahydro-benzo[c]azepine-2-carboxylic acid isopropylamide prepared as a pale oil (64 mg, 99%) from 7-(3-piperidin-1-yl-propoxy)-2,3,4,5-tetrahydro-1H-benzo[c]azepine (50 mg, 0.173 mmol) and isopropyl isocyanate (24 mg, 0.208 mmol) by the method of Procedure D (See herein Example 16), except SCX chromatography was not conducted. ¹H NMR (CDCl₃) δ 7.08 (d, 1H), 6.73 (d, 1H), 6.65 (dd, 1H), 4.32 (s, 2H), 4.15 (d, 1H), 3.98 (t, 2H), 3.85 (m, 1H), 3.67 (m, 2H), 2.89 (m, 2H), 2.46 (t, 2H), 2.40 (m, 4H), 1.96 (m, 2H), 1.78 (m, 2H), 1.59 (qt, 4H), 1.44 (m, 2H), 1.07 (d, 6H); MS (APCI): M+H: 374(100%).

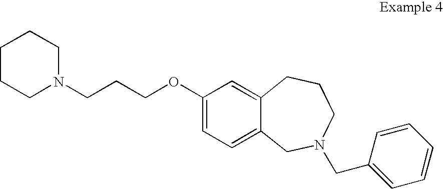

Procedure B: To a stirred solution of 7-(3-piperidin-1-yl-propoxy)-2,3,4,5-tetrahydro-1H-benzo[c]azepine (75 mg, 0.260 mmol) in 10:1 dichloroethane(DCE)/methanol (3.6 mL) containing acetic acid (0.1 equivalent) at room temperature under N₂, is added benzaldehyde (41 mg, 0.386 mmol). After 15 minutes, sodium triacetoxyborohydride (114 mg, 0.54 mmol) is added. Stirring is continued for 30 minutes (or until starting material was consumed by TLC) and the mixture was loaded directly onto a Varian SCX column (10 g). The column was washed with DCM and methanol, and the desired compound was then eluted with a 7N NH₃ in methanol, to provide 2-Benzyl-7-(3-piperidin-1-yl-propoxy)-2,3,4,5-tetrahydro-1H-benzo[c] azepine as a pale oil (54 mg 55%). ¹H NMR (CDCl₃) δ 7.26 (m, 5H), 6.83 (d, 1H), 6.71 (s, 1H), 6.60 (dd, 1H), 3.99 (m, 2H), 3.81 (s, 2H), 3.50 (s, 2H), 3.08 (m, 2H), 2.84 (m, 2H), 2.54 (m, 2H), 2.47 (bs, 4H), 2.02 (m, 2H), 1.74 (bs, 2H), 1.64 (m, 4H), 1.48 (m, 2H); MS (APCI): M+H: 379 (100%).

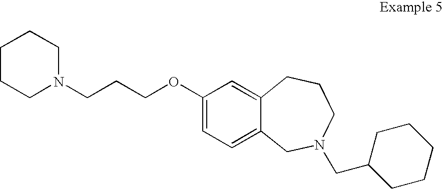

2-Cyclohexylmethyl-7-(3-piperidin-1-yl-propoxy)-2,3,4,5-tetrahydro-1H-benzo[c]azepine; prepared as a pale oil (56 mg, 86%) from 7-(3-piperidin-1-yl-propoxy)-2,3,4,5-tetrahydro-1H-benzo[c]azepine (50 mg, 0.174 mmol) and cyclohexanecarboxaldehyde (30 mg, 0.260 mmol) by the method of Procedure B. (See herein Example 4). ¹H NMR CDCl₃) δ 6.99 (d, 1H), 6.69 (d, 1H), 6.62 (dd, 1H), 3.99 (t, 2H), 3.87 (s, 2H), 3.11 (m, 2H), 2.84 (m, 2H), 2.59 (t, 2H), 2.53 (bs, 4H), 2.14 (d, 2H), 2.04 (m, 2H), 1.70 (m, 11H), 1.41-1.51 (m, 3H), 1.20-1.28 (m, 3H), 0.83 (m, 2H); MS (APCI): M+H: 385 (100%).

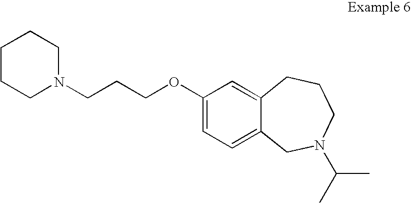

2-Isopropyl-7-(3-piperidin-1-yl-propoxy)-2,3,4,5-tetrahydro-1H-benzo[c]azepine; prepared as a pale oil (67 mg, 78%) from 7-(3-piperidin-1-yl-propoxy)-2,3,4,5-tetrahydro-1H-benzo[c]azepine (75 mg, 0.26 mmol) by the method of Procedure G (see herein Example 21), except that the dihydrochloride was not prepared. ¹H NMR (CDCl₃) δ 7.03 (d, 1H), 6.69 (d, 1H), 6.63 (dd, 1H), 3.97 (t, 2H), 3.77 (s, 2H), 3.08 (t, 2H), 2.82 (m, 2H), 2.78 (sept, 1H), 2.48 (t, 2H), 2.42 (bs, 4H), 1.98 (t, 1H), 1.95 (t, 1H), 1.75 (m, 2H), 1.60 (m, 4H), 1.45 (m, 2H), 1.09 (d, 3H), 1.08 (d, 3H); MS (APCI): M+H: 331 (46%).

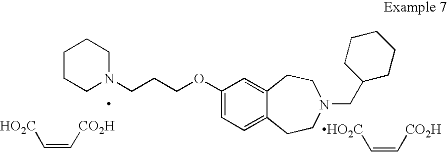

3-Cyclohexylmethyl-7-(3-piperidin-1-yl-propoxy)-2,3,4,5-tetrahydro-1H-benzo[d]azepine dimaleate; prepared by the method of Procedure B. (See herein Example 4). The free base was converted to its dimaleate (2 equivalents maleic acid in boiling ethyl acetate) and recrystallized from ethanol/ethyl acetate. Drying under high vacuum at 100° C gave the title compound as a white solid (4.0 g). ¹H NMR (free base in CDCl₃) δ 6.97 (d, 1H), 6.65 (d, 1H), 6.62 (dd, 1H), 3.97 (t, 2H), 2.84 (m, 4H), 2.57 (m, 4H), 2.46 (t, 2H), 2.40 (bs, 4H), 2.22 (d, 2H), 1.95 (m, 2H), 1.80 (bd, 2H), 1.70 (bm, 3H), 1.59 (m, 4H), 1.50 (m, 1H), 1.44 (m, 2H), 1.14-1.28 (m, 3H), 0.88 (m, 2H); MS (APCI), M+H: 385 (100%).

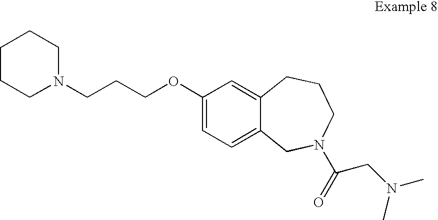

2-Dimethylamino-1-[7-(3-piperidin-1-yl-propoxy)-1,3,4,5-tetrahydro-benzo[c]azepin-2-yl]-ethanone; prepared as a pale oil (12 mg, 18%) from 7-(3-piperidin-1-yl-propoxy)-2,3,4,5-tetrahydro-1H-benzo[c]azepine (50 mg, 0.173 mmol) and N,N-dimethylglycine (22 mg, 0.208 mmol) by the method of Procedure J (see herein Example 28). ¹H NMR (CDCl₃) δ 7.26 (d, 0.5H), 7.03 (d, 0.5H), 6.73 (d, 0.5H), 6.69 (d, 0.5H), 6.65 (m, 1H), 4.65 (s, 1H), 4.49 (bs, 1H), 3.97 (q, 2H), 3.79 (bm, 2H), 3.11 (s, 1H), 3.03 (s, 1H), 2.90 (m, 2H), 2.46 (m, 2H), 2.39 (bs, 4H), 2.31 (s, 3H), 2.21 (s, 3H), 1.95 (m, 2H), 1.81 (m, 2H), 1.59 (m, 4H). 1.44 (m, 2H); MS (APCI), M+H: 374 (100%).

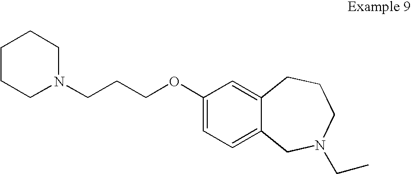

2-Ethyl-7-(3-piperidin-1-yl-propoxy)-2,3,4,5-tetrahydro-1H-benzo[c]azepine; prepared as a pale oil (31 mg, 94%) from 7-(3-piperidin-1-yl-propoxy)-2,3,4,5-tetrahydro-1H-benzo[c]azepine (30 mg, 0.104 mmol) and acetaldehyde (excess) by the method of Procedure B, (See herein Example 4) except that flash chromatography was not performed. H NMR (CDCl₃) δ 7.02 (d, 1H), 6.69 (d, 1H), 6.62 (dd, 1H), 3.97 (t, 2H), 3.84 (s, 2H), 3.09 (m, 2H), 2.83 (m,2H), 2.39-2.49 (m, 8H), 1.96 (m, 2H), 1.72 (m, 2H), 1.59 (m, 4H), 1.44 (m, 2H),1.07 (t, 3H); MS (APCI), M+H: 317 (100%).

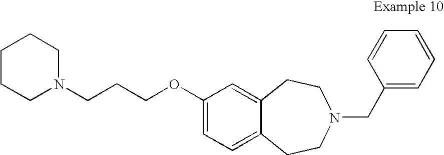

3-Benzyl-7-(3-piperidin-1-yl-propoxy)-2,3,4,5-tetrahydro-1H-benzo[d]azepine; prepared as a pale oil (56 mg, 86%) from 7-(3-piperidin-1-yl-propoxy)-2,3,4,5-tetrahydro-1H-benzo[d]azepine (50 mg, 0.174 mmol) and benzaldehyde (28 mg, 0.264 mmol) by the method of Procedure B. (See herein Example 4). ¹H NMR (CDCl₃) δ 7.30-7.36 (m, 4H1), 7.22-7.27 (m, 1H), 6.96 (d, 1H), 6.63 (d, 1H), 6.61 (dd, 1H), 3.96 (t, 2H), 3.63 (s, 2H), 2.85 (m, 4H), 2.61 (m, 4H), 2.52 (t, 2H), 2.46 (bs, 4H), 1.99 (m, 2H), 1.63 (m, 4H), 1.45 (m, 2H); MS (APCI): M+H: 379 (100%).

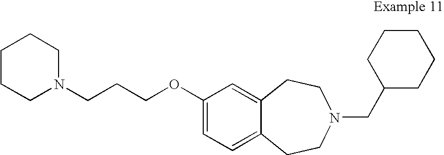

3-Cyclohexylmethyl-7-(3-piperidin-1-yl-propoxy)-2,3,4,5-tetrahydro-1H-benzo[d]azepine; prepared as a pale oil (44 mg, 66%) from 7-(3-piperidin-1-yl-propoxy)-2,3,4,5-tetrahydro-1H-benzo[d]azepine (50 mg, 0.174 mmol) and cyclohexanecarboxaldehyde (29 mg, 0.259 mmol) by the method of Procedure B. (See herein Example 4). ¹H N (CDCl₃) δ 6.97 (d, 1H), 6.65 (d, 1H), 6.61 (dd, 1H), 3.97 (t, 2H), 2.84 (m, 4H), 2.52-2.62 (m, 6H), 2.48 (bs, 4H), 2.24 (d, 2H), 2.01 (m, 2H), 1.80 (bd, 2H), 1.62-1.74 (m, 7H), 1.44-1.56 (m, 3H), 1.12-1.30 (m, 3H), 0.88 (m, 2H); MS (APCI), M+H: 385 (100%).

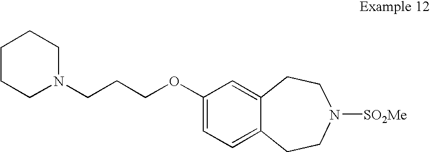

3-Methanesulfonyl-7-(3-piperidin-1-yl-propoxy)-2,3,4,5-tetrahydro-1H-benzo[d]azepine; prepared as a pale solid (49 mg, 77%) from 7-(3-piperidin-1-yl-propoxy)-2,3,4,5-tetrahydro-1H-benzo[d]azepine (50 mg, 0.173 mmol) and methanesulfonyl chloride (24 mg, 0.208 mmol) by the method of Procedure D (see herein Example 16), except flash chromatography was not conducted. ¹H NMR (CDCl₃) δ 7.03 (d, 1H), 6.69 (d, 1H), 6.68 (dd, 1H), 3.99 (t, 2H), 3.42 (m, 4H), 2.97 (m, 4H), 2.77 (s, 3H), 2.55 (t, 2H), 2.49 (bs, 4H), 2.02 (m, 2H), 1.65 (m, 4H), 1.47 (m, 2H); MS (APCI), M+H: 367 (100%).

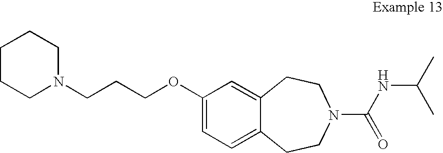

7-(3-Piperidin-1-yl-propoxy)-1,2,4,5-tetrahydro-benzo[d]azepine-3-carboxylic acid isopropylamide; Procedure C: To a stirred mixture 7-(3-piperidin-1-yl-propoxy)-2,3,4,5-tetrahydro-1H-benzo[d]azepine (50 mg, 0.173 mmol) at room temperature in dry DCM (4 mL) under dry N₂ was added isopropyl isocyanate (18 mg, 0.208 mmol) and stirring was continued overnight. PS-Trisamine (Argonaut, 4.46 mmol/g, 200 mg, 0.892 mmol) was added and stirring was continued for several hours. The mixture was suction filtered, the scavenger was rinsed with DCM, and the combined filtrates were concentrated in vacuo. The crude material was loaded onto a Varian SCX column (10 g), the column was washed with DCM and methanol, and the desired compound was then eluted with 7N NH₃ in methanol to provide the title compound as a pale oil (58 mg, 90%). ¹H NMR (CDCl₃) δ 6.99 (d, 1H), 6.67 (d, 1H), 6.64 (dd, 1H), 4.26 (d, 1H), 4.02 (m, 1H), 3.98 (t, 2H), 3.53 (m, 2H), 3.49 (m, 2H), 2.87 (m, 4H), 2.55 (t, 2H), 2.48 (bs, 4), 2.02 (m, 2H), 1.65 (m, 4H), 1.47 (m, 2H), 1.18 (d, 6H); MS (APCI), M+H: 374 (100%).

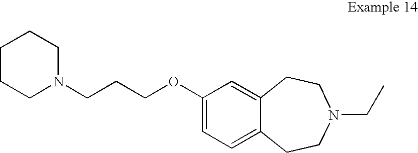

3-Ethyl-7-(3-piperidin-1-yl-propoxy)-2,3,4,5-tetrahydro-1H-benzo[d]azepine; prepared as a pale oil (23 mg, 70%) from 7-(3-piperidin-1-yl-propoxy)-2,3,4,5-tetrahydro-1H-benzo[d]azepine (30 mg, 0.104 mmol) and acetaldehyde (excess) by the method of Procedure B, (See herein Example 4) except that flash chromatography was not performed. ¹H NMR (CDCl₃) δ 6.98 (d, 1H), 6.66 (d, 1H), 6.63 (dd, 1H), 3.98 (t, 2H), 2.89 (m, 4H), 2.66 (m, 4H), 2.60 (q, 2H), 2.51 (t, 2H), 2.45 (bs, 4H), 1.99 (m, 2H), 1.62 (m, 4H), 1.46 (m, 2H), 1.11 (t, 3H); MS (APCI), M+H: 317 (100%).

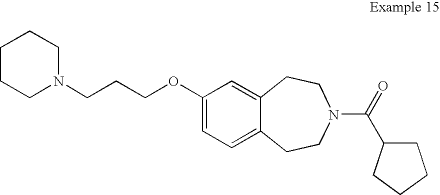

Cyclopentyl-[7-(3-piperidin-1-yl-propoxy)-1,2,4,5-tetrahydro-benzo[d]azepin-3-yl]-methanone; prepared as a pale oil (65 mg, 81%) from 7-(3-piperidin-1-yl-propoxy)-2,3,4,5-tetrahydro-1H-benzo[d]azepine (60 mg, 0.208 mmol) and cyclopentanecarboxylic acid (30 mg, 0.26 mmol) by the method of Procedure J (see herein Example 28). ¹H NMR (CDCl₃) δ 7.02 (m,1H), 6.67 (m, 2H), 3.98 (t, 2H), 3.71 (m, 2H), 3.63 (m, 2H), 2.97 (qt, 1H), 2.86 (m, 4H), 2.53 (t, 2H), 2.47 (bs, 4H), 2.00 (m, 2H), 1.85 (m, 4H), 1.76 (m, 2H), 1.62 (m, 6H), 1.46 (m, 2H); MS (APCI), M+H: 385 (100%).

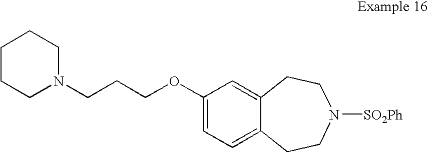

3-Benzenesulfonyl-7-(3-piperidin-1-yl-propoxy)-2,3,4,5-tetrahydro-1H-benzo[d]azepine; Procedure D: To a stirred mixture 7-(3-piperidin-1-yl-propoxy)-2,3,4,5-tetrahydro-1H-benzo[d]azepine (30 mg, 0.104 mmol) PS-DMAP (Argonaut, 1.48 mmol/g, 14 mg, 0.021 mmol), and PS-DIEA (Argonaut, 3.83 mmol/g, 81 mg, 0.312 mmol), at room temperature in dry DCM (4 mL) under N₂ was added benzenesulfonyl chloride (47 mg, 0.268 mmol) and stirring was continued overnight. PS-Trisamine (Argonaut, 4.46 mmol/g, 200 mg, 0.892 mmol) was added and stirring was continued for several hours. The mixture was suction filtered, the scavenger was rinsed with DCM, and the combined filtrates were concentrated in vacuo. The crude material was loaded onto a Varian SCX column (10 g), the column was washed with DCM and methanol, and the desired compound was then eluted with 7N NH₃ in methanol. Further purification by flash chromatography on silica gel (20:1 DCM/7N NH₃ in methanol) furnished the title compound (38 mg, 85%). ¹H NMR (CDCl₃) δ 7.75 (m, 2H), 7.45-7.53 (m, 3H), 6.96 (d, 1H), 6.61 (m, 2H), 3.94 (t,

2H), 3.29 (m, 4H), 2.94 (m, 4H), 2.51 (t, 2H), 2.45 (bs, 4H), 1.98 (m, 2H), 1.63 (m, 4H), 1.46 (m, 2H); MS (APCI), M+H: 429 (100%).

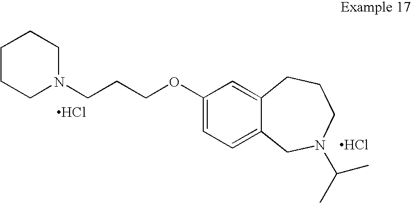

2-Isopropyl-7-(3-piperidin-1-yl-propoxy)-2,3,4,5-tetrahydro-1H-benzo[c]azepine dihydrochloride; prepared by the method of Procedure G. (See herein Example 21). The free base was converted to its dihydrochloride (excess 2M HCl in ether/DCM) to provide the title compound as a white solid (5.5 g). ¹H NMR (free base in CDCl₃) δ 7.03 (d, 1H), 6.69 (d, 1H), 6.62 (dd, 1H), 3.97 (t, 2H), 3.74 (s, 2H), 3.06 (t, 2H), 2.83 (m, 2H), 2.77 (sept, 1H), 2.46 (t, 2H), 2.40 (bs, 4H), 1.96 (m, 2H), 1.74 (m, 2H), 1.59 (m, 4H), 1.45 (m, 2H), 1.08 (d, 3H), 1.06 (d, 3H); MS (APCI): M+H: 331 (40%).

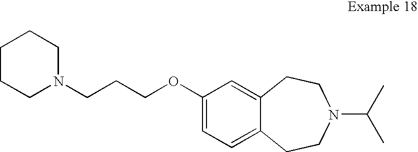

3-Isopropyl-7-(3-piperidin-1-yl-propoxy)-2,3,4,5-tetrahydro-1H-benzo[d]azepine; prepared as a pale oil (56 mg, 97%) from 7-(3-piperidin-1-yl-propoxy)-2,3,4,5-tetrahydro-1H-benzo[d]azepine (50 mg, 0.174 mmol) by the method of Procedure G, (See herein Example 21), except that the dihydrochloride was not prepared. ¹H NMR (CDCl₃) δ 6.98 (d, 1H), 6.67 (d, 1H), 6.63 (dd, 1H), 3.96 (t, 2H), 2.97 (sept, 1H), 2.86 (m, 4H), 2.64 (m, 4H), 2.47 (m, 2H), 2.40 (bs, 4H), 1.96 (m, 2H), 1.59 (m, 4H), 1.43 (m, 2H), 1.03 (d, 6H); MS (APCI), M+H: 331 (100%).

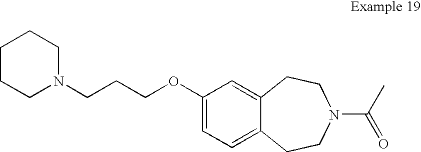

1-[7-(3-Piperidin-1-yl-propoxy)-1,2,4,5-tetrahydro-benzo[d]azepin-3-yl]-ethanone; Procedure E: To a stirred mixture of 7-(3-piperidin-1-yl-propoxy)-2,3,4,5-tetrahydro-1H-benzo[d]azepine (35 mg, 0.121 mmol), PS-DMAP (Argonaut, 1.48 mmol/g, 16 mg, 0.02 mmol), PS-DIEA (Argonaut, 3.83 mmol/g, 138 mg, 0.53 mmol) and triethylamine (2.3 μL, 0.016 mmol) in dry DCM (3.5 mL) at room temperature under dry N₂ was added acetic anhydride (16 mg, 0.158 mmol). After 2 hours, triamine-3 (Silicycle, 1.42 mmol/g, 345 mg, 0.490 mmol) and isocyanate-3 (Silicycle, 1.21 mmol/g, 400 mg, 0.48 mmol) are added and stirring was continued for several hours. The mixture was suction filtered, the scavengers were rinsed with DCM, and the filtrate was concentrated in vacuo. Purification of the residue by flash chromatography on silica (20:1 DCM/7N NH₃ in methanol), gave the title compound as a pale oil (25 mg, 63%). ¹H NMR (CDCl₃) δ 7.00 (m, 1H), 6.60-6.66 (m, 2H), 3.99 (t, 2H), 3.67 (m, 2H), 3.53 (m, 2H), 2.75-2.87 (m, 10H), 2.19 (m, 2H), 2.16 (s, 1.5H), 2.15 (s, 1.5H), 1.83 (m, 4H), 1.54 (bs, 2H); MS (APCI), M+H: 331 (100%).

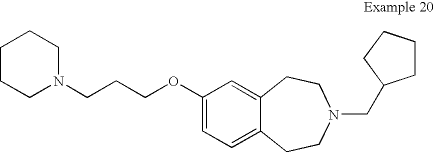

3-Cyclopentylmethyl-7-(3-piperidin-1-yl-propoxy)-2,3,4,5-tetrahydro-1H-benzo[d]azepine; Procedure F: A stirred solution of cyclopentyl-[7-(3-piperidin-1-yl-propoxy)-1,2,4,5-tetrahydro-benzo[d]azepin-3-yl]-methanone (40 mg, 0.104 mmol) and lithium aluminum hydride (1M in THF, 0.21 mL, 0.21 mmol) in dry THF (5 mL) under dyr N₂ was refluxed for 3 hours, cooled to 0° C., and quenched cautiously with excess sodium sulfate decahydrate. After stirring for 1-2 additional hours, the mixture was filtered with suction, the precipitated salts were washed with additional THF, and the combined filtrates concentrated in vacuo. The residue was loaded directly onto a Varian SCX column (10 g). The column was washed with DCM and methanol, and the desired compound was then eluted with 7N NH₃ in methanol. Further purification by flash chromatography on silica gel or preparative TLC (20:1 DCM/7N NH₃ in methanol) afforded the title compound as a pale oil (31 mg, 81%). ¹H NMR (CDCl₃) δ 6.96 (d, 1H), 6.63 (d, 1H), 6.60 (dd, 1H), 3.96 (t, 2H), 2.86 (m, 4H), 2.66 (m, 4H), 2.57 (t, 2H), 2.51 (bs, 4H), 2.44 (d, 2H), 2.09 (sept, 1H), 2.03 (m, 2H), 1.75 (m, 2H), 1.66 (m, 4H), 1.44-1.61 (m, 6H), 1.19 (m, 2H); MS (APCI), M+H: 371 (100%).

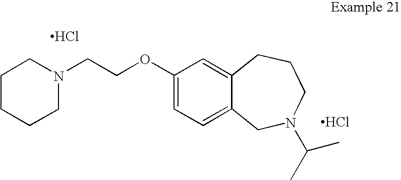

2-Isopropyl-7-(2-piperidin-1-yl-ethoxy)-2,3,4,5-tetrahydro-1H-benzo[c]azepine, dihydrochloride; Procedure G: A stirred solution of 7-(2-piperidin-1-yl-ethoxy)-2,3,4,5-tetrahydro-1H-benzo[c]azepine dihydrochloride (70 mg, 0.202 mmol), acetone (1 mL), and sodium cyanoborohydride (40 mg, 0.636 mmol) in 1:1 DCE/methanol containing acetic acid (3 drops) was heated to 50° C. in a sealed tube overnight. After cooling to room temperature, the mixture was loaded directly onto a Varian SCX column (10 g). The column was washed with DCM and methanol, and the desired compound was then eluted with 7N NH₃ in methanol. This material was converted to its dihydrochloride (2M HCl in ether/DCM), which was isolated as pale yellow solid (46 mg, 59%): ¹H NMR (free base in CDCl₃) δ 7.05 (d, 1H), 6.71 (d, 1H), 6.64 (d, 1H), 4.09 (t, 2H), 3.81 (s, 2H), 3.11 (m, 2H, 2.83 (m, 2H), 2.81 (m, 1H), 2.76 (m, 2H), 2.51 (bs, 4H), 1.77 (bm, 2H), 1.61 (m, 4H), 1.45 (bm, 2H), 1.11 (d, 6H); MS (APCI), M+H: 317 (100%).

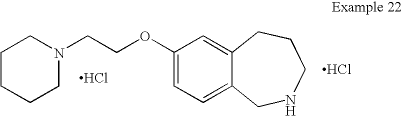

7-(2-Piperidin-1-yl-ethoxy)-2,3,4,5-tetrahydro-1H-benzo[c]azepine dihydrochloride; Procedure H: To a stirred solution of 7-(2-piperidin-1-yl-ethoxy)-1,3,4,5-tetrahydro-benzo[c]azepine-2-carboxylic acid tert-butyl ester (113 mg, 0.302 mmol) in DCM (2 mL) was added 4M HCl in dioxane (1 mL, 4.0 mmol) at room temperature. After 2 hours or until starting material was consumed by TLC, the mixture was concentrated in vacuo. The crude material was twice dissolved in dry methanol and concentrated in vacuo, and the solid was triturated with ether, filtered, and dried. The title compound was obtained as a pale yellow solid (90 mg, 86%). ¹H NMR (free base in CDCl₃) δ 7.04 (bd, 1H), 6.74 (d, 1H), 6.63 (d, 1H), 4.10 (t, 2H), 3.90 (bs, 2H), 3.19 (bs, 2H), 2.89 (bm, 2H), 2.78 (bm, 2H), 2.53 (bs, 4H), 1.75 (bs, 2H), 1.63 (bm, 4H), 1.46 (bm, 2H); MS (APCI), M+H: 275 (100%).

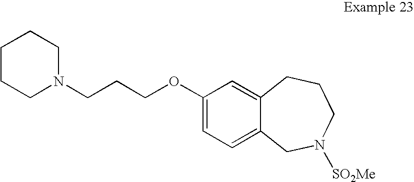

2-Methanesulfonyl-7-(3-piperidin-1-yl-propoxy)-2,3,4,5-tetrahydro-1H-benzo[c]azepine; prepared as a pale oil (72 mg, 96%) from 7-(3-piperidin-1-yl-propoxy)-2,3,4,5-tetrahydro-1H-benzo[c]azepine (60 mg, 0.208 mmol) and methanesulfonyl chloride (24 mg, 0.208 mmol) by the method of Procedure D (see herein Example 16), except SCX chromatography was not conducted. ¹H NMR (CDCl₃) δ 7.08(d, 1H), 6.73 (d, 1H), 6.65 (dd, 1H), 4.46 (s, 2H), 4.03 (t, 2H), 3.74 (m, 2H), 2.95 (m, 2H), 2.86 (m, 6H), 2.48 (s, 3H), 2.25 (m, 2H), 1.84-1.91 (m, 6H), 1.58 (bs, 2H); MS (APCI): M+H: 367 (100%)

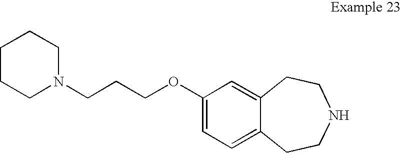

7-(3-Piperidin-1-yl-propoxy)-2,3,4,5-tetrahydro-1H-benzo[d]azepine; prepared as the dihydrochloride (2.6 g, 87%) from 7-(3-piperidin-1-yl-propoxy)-1,3,4,5-tetrahydro-benzo[d]azepine-2-carboxylic acid tert-butyl ester (3.2 g, 8.25 mmol) by the method of Procedure H (see herein Example 22). A portion was free based (aqueous saturated sodium bicarbonate/DCM) to provide the title compound as a pale oil. ¹H NMR (CDCl₃) δ 7.00 (d, 1H), 6.68 (d, 1H), 6.65 (dd, 1H), 4.91 (bs, 1H) 3.98 (t, 2H), 3.04 (m, 4H), 2.95 (m, 4H), 2.51 (t, 2H), 2.44 (bs, 4H), 1.99 (m, 2H), 1.62 (m, 4H), 1.45 (m, 2H); MS (APCI), M+H: 289 (100%).

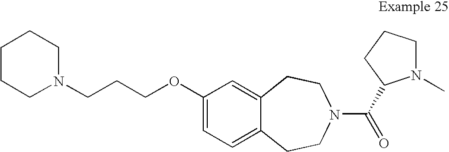

(S)-(1-Methyl-pyrrolidin-2-yl)-[7-(3-piperidin-1-yl-propoxy)-1,2,4,5-tetrahydro-benzo[d]azepin-3-yl]-methanone; prepared as a pale oil (40 mg, 48%) from 7-(3-piperidin-1-yl-propoxy)-2,3,4,5-tetrahydro-1H-benzo[d]azepine (60 mg, 0.208 mmol) and N-methyl-L-proline (54 mg, 0.416 mmol) by the method of Procedure J (see herein Example 28). ¹H NMR (CDCl₃) δ 6.96-7.02 (m, 1H), 6.61-6.69 (m, 2H), 3.95 (t, 2H), 3.60-3.78 (m, 4H), 3.15 (m, 1H), 3.09 (t, 1H), 2.83 (m, 4H), 2.44 (t, 2H), 2.37 (bs, 4H), 2.30 (s, 3H), 2.22 (m, 1H), 2.05-2.14 (m, 1H), 1.73-1.99 (m, 5H); 1.57 (m, 4H), 1.41 (m, 2H); MS (APCI), M+H: 400 (100%).

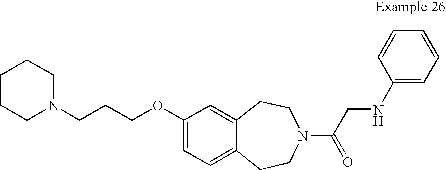

2-Phenylamino-1-[7-(3-piperidin-1-yl-propoxy)-1,2,4,5-tetrahydro-benzo[d]azepin-3-yl]-ethanone; prepared as a pale oil (50 mg, 57%) from 7-(3-piperidin-1-yl-propoxy)-2,3,4,5-tetrahydro-1H-benzo[d]azepine (60 mg, 0.208 mmol) and N-phenylglycine (39 mg, 0.26 mmol) by the method of Procedure J (see herein Example 28). ¹H NMR (CDCl₃) δ 9.07 (bs, 0.25 H), 7.54 (d, 0.75H), 7.30 (t, 0.75H), 7.07-7.15 (m, 1.5H), 6.97 (m, 1H), 6.56-6.67 (m, 4H), 4.89 (m, 0.75H), 4.06 (m, 0.75H), 3.88-3.94 (m, 3H), 3.71 (m, 2H), 3.49 (m, 1.5H), 2.97 (m, 0.75H), 2.79-2.89 (m, 3.25H), 2.52 (t, 2H), 2.46 (bs, 4H), 1.98 (m, 2H), 1.61 (m, 4H), 1.41 (m, 2H); MS (APCI), M+H: 422 (100%).

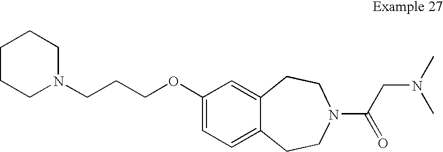

2-Dimethylamino-1-[7-(3-piperidin-1-yl-propoxy)-1,2,4,5-tetrahydro-benzo[d]azepin-3-yl]-ethanone; prepared as a pale oil (47 mg, 60%) from 7-(3-piperidin-1-yl-propoxy)-2,3,4,5-tetrahydro-1H-benzo[d]azepine (60 mg, 0.208 mmol) and N,N-dimethylglycine (54 mg, 0.52 mmol) by the method of Procedure J (see herein Example 28). ¹H NMR (CDCl₃) δ 6.99 (m, 1H), 6.61-6.68 (m, 2H), 3.95 (t, 2H), 3.62-3.70 (m, 4H), 3.16 (s, 2H), 2.84 (m, 4H), 2.45 (t, 2H), 2.38 (bs, 4H), 2.27 (s, 6H), 1.94 (m, 2H), 1.57 (m, 4H), 1.41 (m, 2H); MS (APCI), M+H: 374 (100%).

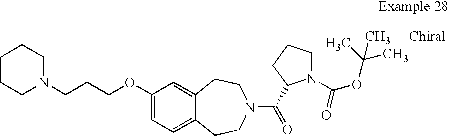

(S)-2-[7-(3-Piperidin-1-yl-propoxy)-1,2,4,5-tetrahydro-benzo[a]azepine-3-carbonyl]-pyrrolidine-1-carboxylic acid tert-butyl ester; Procedure J: A mixture of 7-(3-piperidin-1-yl-propoxy)-2,3,4,5-tetrahydro-1H-benzo[d]azepine (70 mg, 0.242 mmol), N-(tert-butoxycarbonyl)-L-proline (104 mg, 0.485 mmol), PS-Carbodiimide (Argonaut, 1.32 mmol/g, 367 mg, 0.485 mmol) and 1-hydroxybenzotriazole (HOBt) (49 mg, 0.363 mmol) in dry 1:1 DCM/DMF (10 mL) under dry N₂ was stirred at room temperature overnight. PS-Trisamine (Argonaut, 4.46 mmol/g, 480 mg, 2.14 mmol) was added and stirring was continued for several hours. The mixture was suction filtered, the scavenger was rinsed with DCM, and the combined filtrates were concentrated in vacuo. The crude material was loaded onto a Varian SCX column (10 g), the column was washed with DCM and methanol, and the desired compound was eluted with 7N NH₃ in methanol. Further purification by flash chromatography on silica gel (20:1 DCM/7N NH₃ in methanol) provided the title compound as a pale oil (77 mg, 66%). ¹H NMR (CDCl₃) δ 6.91-6.98 (m, 1H), 6.56-6.63 (m, 2H), 4.70 (m, 0.5H), 4.55 (dd, 0.5), 3.91 (m, 2H), 3.15-3.75 (m, 6H), 2.70-3.12 (m, 4H), 2.53 (m, 2H), 2.47 (bs, 4H), 1.86-2.18 (m, 4H), 1.78 (m, 2H), 1.62 (m, 4H), 1.32-1.41 (m, 11H); MS (APCI), M+H−100: 386 (100%).

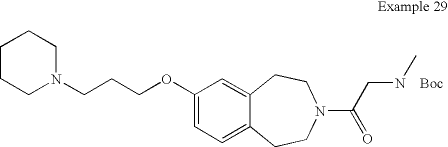

Methyl-{2-oxo-2-[7-(3-piperidin-1-yl-propoxy)-1,2,4,5-tetrahydro-benzo[d]azepin-3-yl]-ethyl)}-carbamic acid tert-butyl ester, prepared as a pale oil (60 mg, 54%) from 7-(3-piperidin-1-yl-propoxy)-2,3,4,5-tetrahydro-1H-benzo[d]azepine (70 mg, 0.242 mmol) and N-(tert-butoxycarbonyl)-sarcosine (104 mg, 0.485 mmol) by the method of Procedure J (see herein Example 28). ¹H NMR (CDCl₃) δ 7.01 (m, 1H), 6.67 (m, 2H), 4.14 (s, 1.3H), 4.06 (s, 0.7H), 3.98 (t, 2H), 3.69 (m, 2H), 3.52 (m, 2H), 2.93 (s, 3H), 2.86 (m, 4H), 2.56-2.62 (t, 2H), 2.49-2.56 (bs, 4H), 2.02-2.06 (m, 2H), 1.65-1.70 (m, 4H), 1.45-1.49 (m, 5.9H), 1.44 (s, 3.1H), 1.48 (m, 2H); MS (APCI), M+H: 460 (100%).

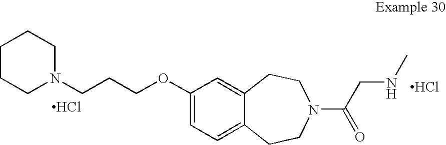

2-Methylamino-1-[7-(3-piperidin-1-yl-propoxy)-1,2,4,5-tetrahydro-benzo[d]azepin-3-yl]-ethanone dihydrochloride; prepared as a pale oil (35 mg, 68%) from methyl-{2-oxo-2-[7-(3-piperidin-1-yl-propoxy)-1,2,4,5-tetrahydro-benzo[d]azepin-3-yl]-ethyl}-carbamic acid tert-butyl ester (55 mg, 0.12 mmol) by the method of Procedure H (see herein Example 22). ¹H NMR (CD₃OD) δ 7.08 (m, 1H), 6.76 (m, 2H), 4.15 (bm, 2H), 4.08 (bm, 2H), 3.69 (m, 2H), 3.57 (m, 4H), 3.31 (m, 2H), 2.98 (bm, 4H), 2.88 (bm, 2H), 2.75 (s, 3H), 2.25 (bs, 2H), 1.95 (bm, 2H), 1.85 (bm, 3H), 1.56 (bm, 1H); MS (APCI), M+H: 360 (100%).

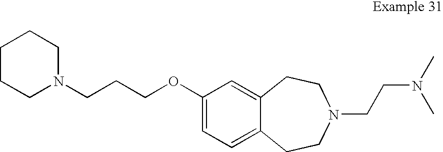

Dimethyl-{2-[7-(3-piperidin-1-yl-propoxy)-1,2,4,5-tetrahydro-benzo[d]azepin-3-yl]-ethyl}-amine; prepared as a pale oil (26 mg, 87%) from 2-dimethylamino-1-[7-(3-piperidin-1-yl-propoxy)-1,2,4,5-tetrahydro-benzo[d]azepin-3-yl]-ethanone (31 mg, 0.083 mmol) by the method of Procedure F (see herein Example 20). Flash chromatography was performed twice. ¹H NMR (CDCl₃) δ 6.97 (d, 1H), 6.63 (m, 2H), 3.96 (t, 2H), 2.85 (m, 4H), 2.64 (m, 6H), 2.45 (m, 4H), 2.39 (bs, 4H), 2.25 (s, 6H), 1.95 (m, 2H), 1.58 (m, 4H), 1.43 (m, 2H); MS (APCI), M+H: 360 (100%).

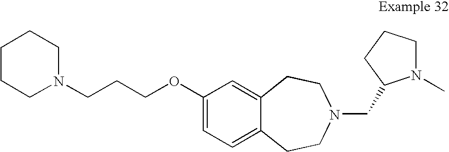

(S)-3-(1-Methyl-pyrrolidin-2-ylmethyl)-7-(3-piperidin-1-yl-propoxy)-2,3,4,5-tetrahydro-1H-benzo[d]azepine; prepared as a pale oil (11 mg, 45%) from (S)-(1-methyl-pyrrolidin-2-yl)-[7-(3-piperidin-1-yl-propoxy)-1,2,4,5-tetrahydro-benzo[d]azepin-3-yl]-methanone (25 mg, 0.063 mmol) by the method of Procedure F (see herein Example 20). Flash chromatography was performed twice. ¹H NMR (CDCl₃) δ 6.96 (d, 1H), 6.65 (bs, 1H), 6.63 (dd, 1H), 3.97 (t, 2H), 3.06 (t, 1H), 2.84 (m, 4H), 2.61-2.71 (m, 5H), 2.31-2.48 (m, 8H), 2.43 (s, 3H), 2.18 (m, 1H), 1.92-2.05 (m, 3H), 1.68-1.84 (m, 2H), 1.56 (m, 5H), 1.43 (m, 2H); MS (APCI), M+H: 386 (100%).

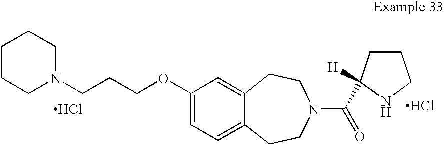

[7-(3-Piperidin-1-yl-propoxy)-1,2,4,5-tetrahydro-benzo[d]azepin-3-yl]-pyrrolidin-2-yl-methanone dihydrochloride; prepared as a pale oil (50 mg, 80%) from 2-[7-(3-piperidin-1-yl-propoxy)-1,2,4,5-tetrahydro-benzo[d]azepine-3-carbonyl]-pyrrolidine-1-carboxylic acid tert-butyl ester (67 mg, 0.138 mmol) by the method of Procedure H (see herein Example 22). ¹H NMR (CDCl₃) δ 12.01 (s, 1H), 11.64 (s, 1H), 7.82 (bd, 1H), 7.02 (d, 1H), 6.68 (bd, 1H), 6.65 (bs, 1H), 4.83 (bs, 1H), 4.07 (bs, 2H), 3.89 (m, 1H), 3.60 (bs, 4H), 3.50 (bm, 2H), 3.19 (bs, 2H), 3.00 (bm, 1H), 2.85 (bm, 3H), 2.70 (bm, 2H), 2.54 (bm, 1H), 2.43 (bs, 2H), 2.31 (bm, 2H), 2.19 (bm, 1H), 2.06 (bm, 1H), 1.89 (bm, 5H), 1.43 (bm, 1H); MS (APCI), M+H: 386(100%).

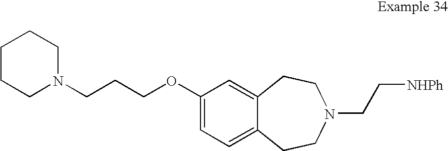

Phenyl-(2-[7-(3-piperidin-1-yl-propoxy)-1,2,4,5-tetrahydro-benzo[d]azepin-3-yl]-ethyl}-amine; prepared as a pale oil (9 mg, 51%) from 2-phenylamino-1-[7-(3-piperidin-1-yl-propoxy)-1,2,4,5-tetrahydro-benzo[d]azepin-3-yl]-ethanone (18 mg, 0.043 mmol) by the method of Procedure F (see herein Example 20). Flash chromatography was performed twice. ¹H NMR (CDCl₃) δ 7.20 (t, 2H), 6.98 (d, 1H), 6.62-6.73 (m, 5H), 4.42 (bs, 1H), 3.97 (t, 2H), 3.17 (t, 2H), 2.85 (bm, 4H), 2.74 (t, 2H), 2.65 (bt, 4H), 2.49 (bt, 2H), 2.41 (bs, 4H), 1.96 (m, 2H), 1.60 (m, 4H), 1.44 (m, 2H); MS (APCI), M+H: 408 (100%). 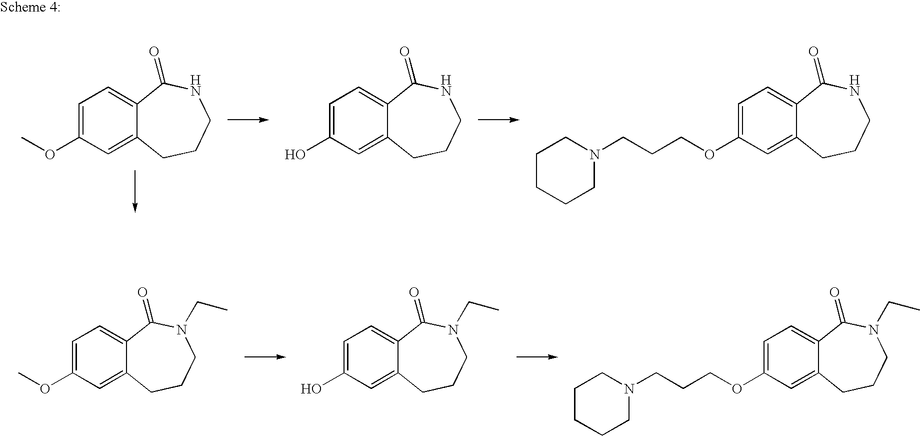 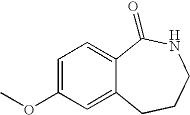
Preparation 7.

7-Methoxy-2,3,4,5-tetrahydro-benzo[c]azepin-1-one is prepared by the procedures similar to those described in Shtacher, G.; Erez, M.; Cohen, S. J Med Chem 1973, 16, 516. 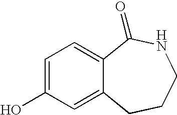
Preparation 8.

7-Hydroxy-2,3,4,5-tetrahydro-benzo[c]azepin-1-one is prepared by the procedures similar to those described in Fisher, M. J. et al; J Med Chem 1999, 42, 4875. 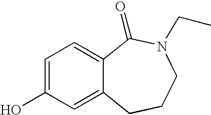
Preparation 9.

### 2-Ethyl-7-hydroxy-2,3,4,5-tetrahydro-benzo[c]azepin-1-one

To a mixture of 7-methoxy-2,3,4,5-tetrahydro-benzo[c]azepin-1-one (0.50 g, 2.6 mmol) in THF (15 mL) is added sodium hydride (60% mineral oil suspension, 150 mg). The suspension is heated at reflux for 1 h, and cooled to room temperature. Ethyl iodide (2.1 mL, 26 mmol) is added, and the mixture is stirred at room temperature overnight. The mixture is partitioned between EtOAc and water. After the aqueous phase is extracted with EtOAc (2×), the combined organic phase is washed with brine and dried (MgSO₄). After removal of the solvent, the residue is purified by flash chromatography (Biotage 40M SiO₂, elute 40% EtOAc:hexane—80% EtOAc:hexane, linear gradient) to yield 2-Ethyl-7-methoxy-2,3,4,5-tetrahydro-benzo[c]azepin-1-one as a colorless oil (0.39 g, 68%). The material is dissolved in CH₂Cl₂ (10 mL) and cooled to −78° C. To the cooled mixture is added a solution of boron tribromide (1 M, 6.2 mL, 6.2 mmol) in CH₂Cl₂. After 0.5 h, the temperature is warmed to 0° C. and stirred for 2 h. After the reaction is carefully quenched with ice, EtOAc and water is added, and the mixture is vigorously stirred overnight. The phases are separated, and the organic phase is extracted with EtOAc (2×). The combined organic phase is washed with brine and dried (MgSO₄). The solvent is removed in vacuo, and the residue is purified by flash chromatography (Biotage 40M SiO₂, elute 40% EtOAc:hexane—80% EtOAc:hexane, linear gradient) to provide 2-Ethyl-7-hydroxy-2,3,4,5-tetrahydro-benzo[c]azepin-1-one (0.135 g, 37%). MS (ES+)

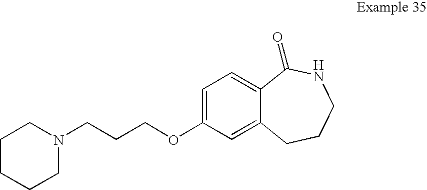

### 7-(3-Piperidin-1-yl-propoxy)-2,3,4,5-tetrahydro-benzo[c]azepin-1-one

Procedure K: A mixture of 7-hydroxy-2,3,4,5-tetrahydro-benzo[c]azepin-1-one (0.38 g, 2.15 mmol, Cs₂CO₃ (1.40 g, 4.3 mmol), KI (35.6 mg, 0.21 mmol), and N-(3-chloropropyl)piperidine (0.42 g, 2.6 mmol) in dioxane (25 mL) is heated at 90° C. for 20 h. The mixture is partitioned between EtOAc and water. The phases are separated, and the aqueous phase is extracted with EtOAc (2×). The combined organic phase is washed with brine, dried (MgSO₄), and concentrated in vacuo. The resulting solid is triturated with petroleum ether and filtered to give the title compound as a white solid (0.34 g, 52%). MS(ES+) 303.4(M+H)⁺.

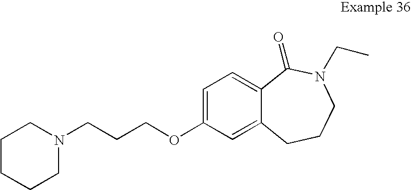

2-Ethyl-7-(3-piperidin-1-yl-propoxy)-2,3,4,5-tetrahydro-benzo[c]azepin-1-one is prepared from 2-ethyl-7-hydroxy-2,3,4,5-tetrahydro-benzo[c]azepin-1-one (0.135 g, 0.66 mmol) in a manner substantially analogous to Procedure K (See herein—Example 35) except DMF is used in place of dioxane. Following aqueous workup, the crude material is purified by chromatography [Varian 10 g SiO2 cartridge, gravity elute with 10% (25/5/1 CHCl3/MeOH NO₄OH)/90% (10% MeOH/CHCl₃)] to obtain the title compound as a colorless oil (0.146 g, 67%). MS (ES+) 331.1.

Utilizing the procedures provided herein, in addition to methods known in the art, compounds of Formula I were prepared. Structural figures for representative examples of Formula I are shown the following pages.

| Example | | Observed |
|---|---|---|
| Number | Structure | Mass |
| 1 | 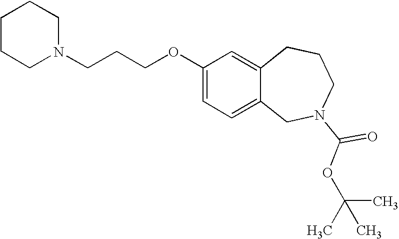 | 389 |
| 2 | 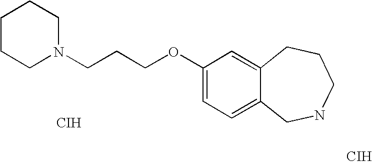 | 289 |
| 3 | 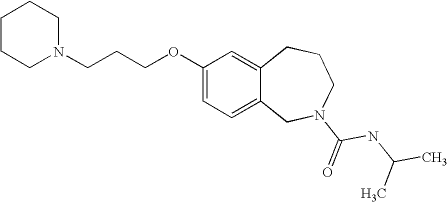 | 374 |
| 4 | 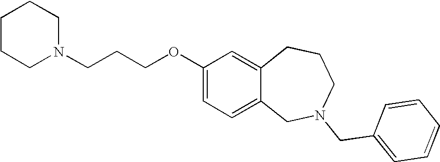 | 379 |
| 5 | 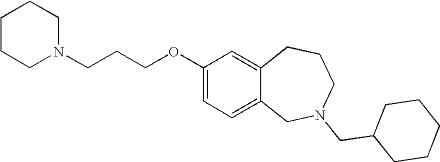 | 385 |
| 6 | 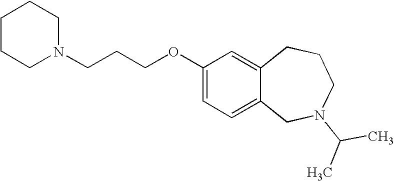 | 331 |
| 7 | 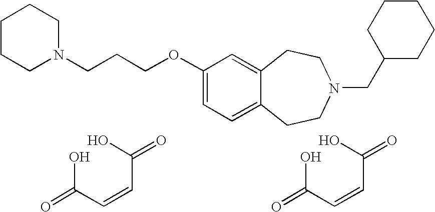 | 385 |
| 8 | 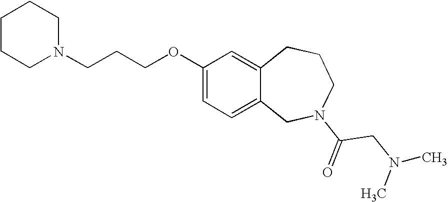 | 374 |
| 9 | 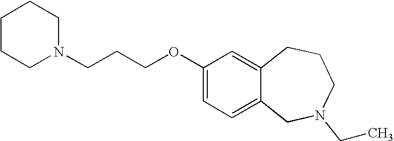 | 317 |
| 10 | 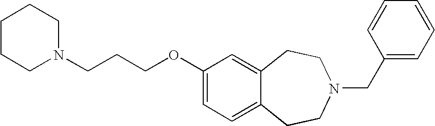 | 379 |
| 11 | 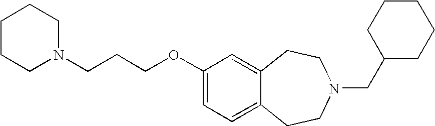 | 385 |
| 12 | 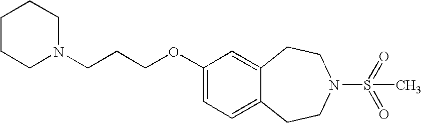 | 367 |
| 13 | 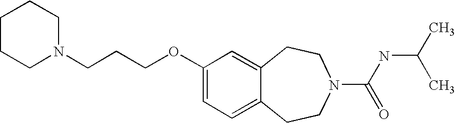 | 374 |
| 14 | 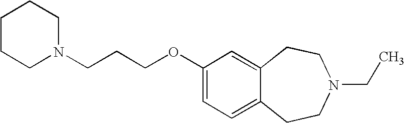 | 317 |
| 15 | 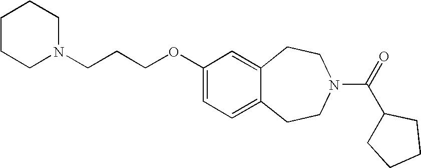 | 385 |
| 16 | 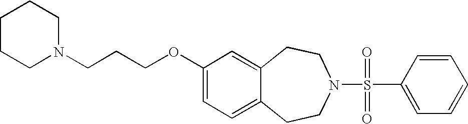 | 429 |
| 17 | 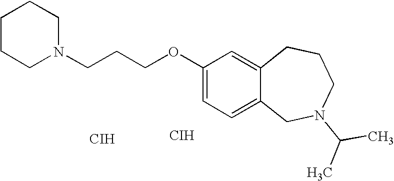 | 331 |
| 18 | 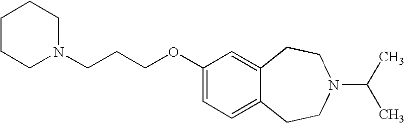 | 331 |
| 19 | 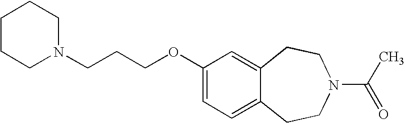 | 331 |
| 20 | 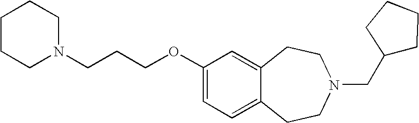 | 371 |
| 21 | 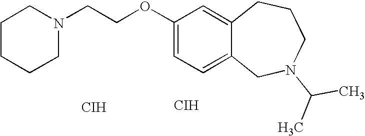 | 317 |
| 22 | 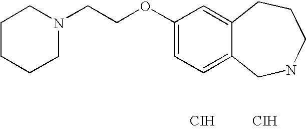 | 275 |
| 23 | 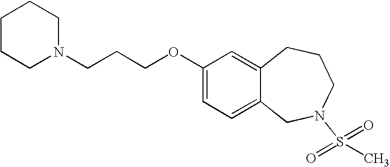 | 367 |
| 24 | 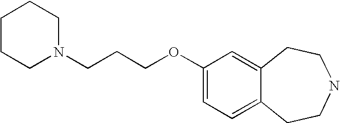 | 289 |
| 25 | 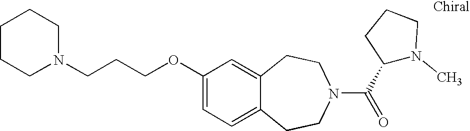 | 400 |
| 26 | 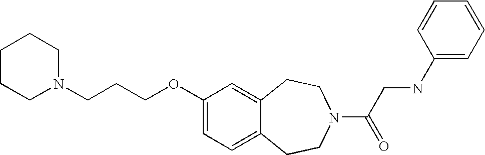 | 422 |
| 27 | 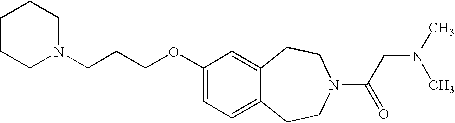 | 374 |
| 28 | 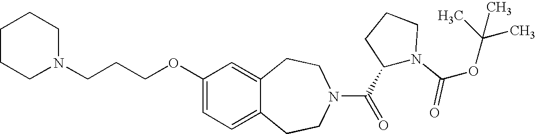 | 386 |
| 29 | 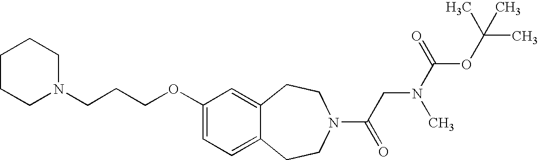 | 460 |
| 30 | 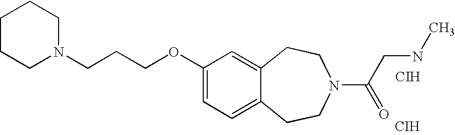 | 360 |
| 31 | 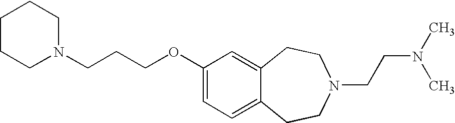 | 360 |
| 32 | 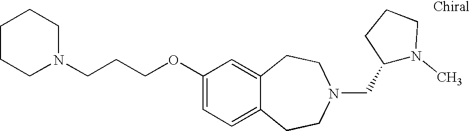 | 386 |
| 33 | 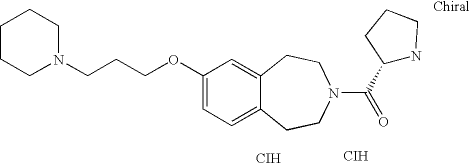 | 386 |
| 34 | 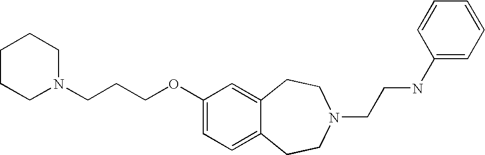 | 408 |
| 35 | 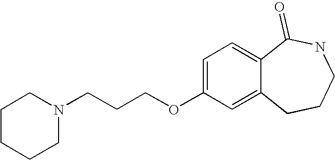 | 303 |
| 36 | 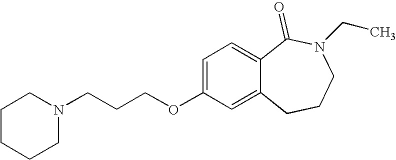 | 331 |

The optimal time for performing the reactions of the Schemes and the Route can be determined by monitoring the progress of the reaction via conventional chromatographic techniques. Furthermore, it is preferred to conduct the reactions of the invention under an inert atmosphere, such as, for example, argon, or, particularly, nitrogen. Choice of solvent is generally not critical so long as the solvent employed is inert to the ongoing reaction and sufficiently solubilizes the reactants to effect the desired reaction. The compounds are preferably isolated and purified before their use in subsequent reactions. Some compounds may crystallize out of the reaction solution during their formation and then collected by filtration, or the reaction solvent may be removed by extraction, evaporation, or decantation. The intermediates and final products of formula I may be further purified, if desired by common techniques such as recrystallization or chromatography over solid supports such as silica gel or alumina.

The skilled artisan will appreciate that not all substituents are compatible with all reaction conditions. These compounds may be protected or modified at a convenient point in the synthesis by methods well known in the art.

The compound of Formula I is preferably formulated in a unit dosage form prior to administration. Therefore, yet another embodiment of the present invention is a pharmaceutical composition comprising a compound of Formula I and one or more pharmaceutically acceptable carriers, diluents or excipients.

The present pharmaceutical compositions are prepared by known procedures using well-known and readily available ingredients. In making the formulations of the present invention, the active ingredient (Formula I compound) will usually be mixed with a carrier, or diluted by a carrier, or enclosed within a carrier which may be in the form of a capsule, sachet, paper or other container. When the carrier serves as a diluent, it may be a solid, semisolid or liquid material that acts as a vehicle, excipient, or medium for the active ingredient. Thus, the compositions can be in the form of tablets, pills, powders, lozenges, sachets, cachets, elixirs, suspensions, emulsions, solutions, syrups, aerosol (as a solid or in a liquid medium), soft and hard gelatin capsules, suppositories, sterile injectable solutions and sterile packaged powders.

Some examples of suitable carriers, excipients, and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methyl and propylhydroxybenzoates, talc, magnesium stearate and mineral oil. The formulations can additionally include lubricating agents, wetting agents, emulsifying and suspending agents, preserving agents, sweetening agents or flavoring agents. The compositions of the invention may be formulated so as to provide quick, sustained or delayed release of the active ingredient after administration to the patient.

The compositions of the present invention may be formulated in sustained release form to provide the rate controlled release of any one or more of the components or active ingredients to optimize the therapeutic effects, i.e., antihistarunic activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injections or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidify.

Also included are solid form preparations which are intended to be converted, shortly before use, to liquid form preparations for either oral or parenteral administration, Such liquid forms include solutions, suspensions and emulsions.

The compounds of the invention may also be deliverable transdermally. The transdermal compositions may take the form of creams, lotions, aerosols and/or emulsions and can be included in a transdermal patch of the matrix or reservoir type as a re conventional in the art for this purpose.

Preferably the compound is administered orally.

Preferably, the pharmaceutical preparation is in a unit dosage form. In such form, the preparation is subdivided into suitably sized unit doses containing appropriate quantities of the active components, e.g., an effective amount to achieve the desired purpose.

The quantity of the inventive active composition in a unit dose of preparation may be generally varied or adjusted from about 0.01 milligrams to about 1,000 milligrams, preferably from about 0.01 to about 950 milligrams, more preferably from about 0.01 to about 500 milligrams, and typically from about 1 to about 250 milligrams, according to the particular application. The actual dosage employed may be varied depending upon the patient's age, sex, weight and severity of the condition being treated. Such techniques are well known to those skilled in the art. Generally, the human oral dosage form containing the active ingredients can be administered 1 or 2 times per day.

Utility

Compounds of Formula I are effective as histamine H3 receptor antagonists. More particularly, these compounds are selective histamine H3 receptor antagonists that have little or no affinity for histamine receptor GPRv53([4R). As selective antagonists, the compounds of Formula I are useful in the treatment of diseases, disorders, or conditions responsive to the inactivation of the histamine H3 receptor, including but not limited to obesity and other eating-related disorders. It is postulated that selective antagonists of H3R will raise brain histamine levels and possibly that of other monoamines resulting in inhibition of food consumption while minimizing peripheral consequences. Although a number of H3R antagonists are known in the art, none have proven to be satisfactory obesity drugs. There is increasing evidence that histamine plays an important role in energy homeostasis. Histamine, acting as a neurotransmitter in the hypothalamus, suppressed appetite. Histamine is an almost ubiquitous amine found in many cell types and it binds to a family of G protein-coupled receptors (GPCRs). This family provides a mechanism by which histamine can elicit distinct cellular responses based on receptor distribution. Both the H1R and H2R are widely distributed. H3R is primarily expressed in the brain, notably in the thalamus and caudate nucleus. High density of expression of H3R was found in feeding center of the brain. A novel histamine receptor GPRv53 has been recently identified. GPRv53 is found in high levels in peripheral white blood cells; only low levels have been identified in the brain by some investigators while others cannot detect it in the brain. However, any drug discovery effort initiated around H3R must consider GPRv53 as well as the other subtypes.

The inventive compounds can readily be evaluated by using a competitive inhibition Scintillation Proximity Assay (SPA) based on a H3R binding assay using [3H] a methylhistamine as ligand. Stable cell lines, including but not limited to HEK can be transfected with cDNA coding for H3R to prepare membranes used for the binding assay. The technique is illustrated below (Preparation of Histamine Receptor Subtype Membranes) for the histamine receptor subtypes.

Membranes isolated as described in (Preparation of Histamine Receptor Subtype Membranes) were used in a [35S]GTPχS functional assay. Binding of [35S]GTPχS to membranes indicates agonist activity. Compounds of the invention of Formula I were tested for their ability to inhibit binding in the presence of agonists. Alternately, the same transfected cell lines were used for a cAMP assay wherein H3R agonists inhibited forskolin-activated synthesis of cAMP. Compounds of Formula I were tested for their ability to permit forskolin-stimulated cAMP synthesis in the presence of agonist.

Preparation of Histamine Receptor Subtype Membranes

A. Preparation H1R Membranes

cDNA for the human histamine 1 receptor (H1R) was cloned into a mammalian expression vector containing the CMV promoter (pcDNA3.1(+), Invitogen) and transfected into HEK293 cells using the FuGENE Tranfection Reagent (Roche Diagnostics Corporation). Transfected cells were selected using G418 (500 μ/ml). Colonies that survived selection were grown and tested for histamine binding to cells grown in 96-well dishes using a scintillation proximity assay (SPA) based radioligand binding assay. Briefly, cells, representing individual selected clones, were grown as confluent monolayers in 96-well dishes (Costar Clear Bottom Plates, #3632) by seeding wells with 25,000 cells and growing for 48 hours (37° C., 5% CO₂). Growth media was removed and wells were rinsed two times with PBS (minus Ca²⁺ or Mg²⁺). For total binding, cells were assayed in a SPA reaction containing 50 mM Tris-HCL (assay buffer), pH 7.6, 1 mg wheat germ agglutinin SPA beads (Amersham Pharmacia Biotech, #RPNQ0001), and 0.8 nM ³H-pyrilamine (Net-594, NEN) (total volume per well=200 ηl). Astemizole (10 μM, Sigma #A6424) was added to appropriate wells to determine non-specific binding. Plates were covered with FasCal and incubated at room temperature for 120 minutes. Following incubation, plates were centrifuged at 1,000 rpm (˜800 g) for 10 minutes at room temperature. Plates were counted in a Wallac Trilux 1450 Microbeta scintillation counter. Several clones were selected as positive for binding, and a single clone (HIR40) was used to prepare membranes for binding studies. Cell pellets, representing ˜10 grams, were resuspended in 30 ml assay buffer, mixed by vortexing, and centrifuged (40,000 g at 4° C.) for 10 minutes. The pellet resuspension, vortexing, and centrifugation was repeated 2 more times. The final cell pellet was resuspended in 30 ml and homogenized with a Polytron Tissue Homogenizer. Protein determinations were done using the Coomassie Plus Protein Assay Reagent (Pierce). Five micrograms of protein was used per well in the SPA receptor-binding assay.

B. Preparation H2R Membranes

cDNA for the human histamine 2 receptor was cloned, expressed and transfected into HEK 293 cells as described above. Histamine binding to cells was assayed by SPA described above. For total binding, cells were assayed in a SPA reaction containing 50 mM Tris-HCl (assay buffer), pH 7.6, 1 mg wheat germ agglutinin SPA beads (Amersham PharmaciaBiotech, #RPNQ0001), and 6.2 nM ³H-tiotidine (Net-688, NEN) (total volume per well=200 μl). Cimetidine (10 μM, Sigma #C4522) was added to appropriate wells to determine non-specific binding.

Several clones were selected as positive for binding, and a single clone (H2R10) was used to prepare membranes for binding studies. Five micrograms of protein was used per well in the SPA receptor-binding assay.

C. Preparation of H3R Membranes

cDNA for the human histamine 3 receptor was cloned and expressed as described in (Preparation of Histamine Receptor Subtye Membranes: A), above. Transfected cells were selected using G418 (500 μ/ml), grown, and tested for histamine binding by the SPA described above. For total binding, cells were assayed in a SPA reaction described above containing 50 mM Tris-HCL (assay buffer), pH 7.6, 1 mg wheat germ agglutinin SPA beads (Amersham Pharmacia Biotech, #RPNQ0001), and 1nM (³M)-n-alpha-methylhistamine (NEN, NET1027) (total volume per well=200μl). Thioperimide was added to determine non-specific binding. Several clones were selected as positive for binding, and a single clone (H3R8) was used to prepare membranes for binding studies described above. Five micrograms of protein was used per well in the SPA receptor-binding assay.

All compounds set forth in examples 1 to 36 exhibited affinity for the H3 receptor greater than 1 uM. Preferred compounds of the invention exhibited affinity for the H3 receptor greater than 200 nM. Most preferred compounds of the invention exhibit affinity for the H3 receptor greater than 20 nM.

D. Preparation of GPRv53 Membranes

cDNA for the human GPRv53 receptor was cloned and expressed as described in (Preparation of Histamine Receptor Subtype Membranes: A), above. Transfected cells were selected, tested for histamine binding, and selected. HEK293 GPRv53 50 cells were grown to confluency in DMEM/F12 (Gibco) supplemented with 5% FBS and 500 ug/ml G418 and washed with Delbecco's PBS (Gibco) and harvested by scraping. Whole cells were homogenized with a Polytron tissuemizer in binding buffer, 50 mM Tris pH 7.5. Cell lysates, 50 ug, were incubated in 96 well dishes with 3 nM (3H) Histamine and compounds in binding buffer for 2 hours at room temperature. Lysates were filtered through glass fiber filters (Perlin Elmer) with a Tomtec cell harverster. Filters were counted with melt-on scintillator sheets (Perlin Elmer) in a Wallac Trilux 1450 Microbeta Scintillation counter for 5 minutes.

Pharmacological Results

cAMP ELISA

HEK293 H3R8 cells prepared as described above were seeded at a density of 50,000 cells/well and grown overnight in DMEM/F12 (Gibco) supplemented with 5% FBS and 500 ug/ml G418. The next day tissue culture medium was removed and replaced with 50 μl cell culture medium containing 4 mM 3-isobutyl-1-methylxanthine (Sigma) and incubated for 20 minutes at room temperature. Antagonist were added in 50 μl cell culture medium and incubated for 20 minutes at room temperature. Agonist R(−)α methylhistamine (RBI) at a dose response from 1×10⁻¹⁰ to 1×10⁻⁵ M was then added to the wells in 50 μl cell culture medium and incubated for 5 minutes at room temperature. Then 50 μl of cell culture medium containing 20 μM Forskolin (Sigma) was added to each well and incubated for 20 minutes at room temperature. Tissue culture medium was removed and cells were lysed in 0.1M HCl and cAMP was measured by ELISA (Assay Designs, Inc.).

[35S] GTP γ [S] Binding Assay

Antagonist activity of selected compounds was tested for inhibition of [35S] GTP γ [S] binding to H3R membranes in the presence of agonists. Assays were run at room temperature in 20 mM HEPES, 100 mM NaCl, 5 mM MgCl₂ and 10 uM GDP at pH 7.4 in a final volume of 200 ul in 96-well Costar plates. Membranes isolated from H3R8-expressing HEK293 cell line (20 ug/well) and GDP were added to each well in a volume of 50 μl assay buffer. Antagonist was then added to the wells in a volume of 50 μl assay buffer and incubated for 15 minutes at room temperature. Agonist R(−)alpha methylhistamine (RBI) at either a dose response from 1×10⁻¹⁰ to 1×10⁻⁵ M or fixed concentration of 100 nM were then added to the wells in a volume of 50 μl assay buffer and incubated for 5 minutes at room temperature. GTP γ [35S] was added to each well in a volume of 50 μl assay buffer at a final concentration of 200 pM, followed by the addition of 50 μl of 20 mg/ml WGA coated SPA beads (Amersham). Plates were counted in Wallac Trilux 1450 Microbeta scintillation counter for 1 minute. Compounds that inhibited more than 50% of the specific binding of radioactive ligand to the receptor were serially diluted to determine a K[i](nM). The results are given below for the indicated compound.

TABLE 1

| Compound | Ki (nM) | Structure |
|---|---|---|
| Example 1 | 5.1 | 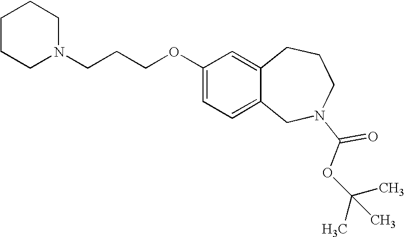 |
| Example 2 | 0.85 | 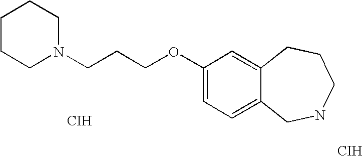 |

To investigate the selectivity of the antagonists for the histamine receptors, a competitive binding assay described above was performed. The ability of example 1 and 2 (structures given above) to selectively inhibit binding to H3R, H1R, H2R and H4R was determined. Importantly, the identification of H3R-specific antagonists that do bind the newly identified H4R was demonstrated. Until the present invention, most known H3R antagonists also bound H4R. As demonstrated in Table 2, example 1 and example 2 did not inhibit binding H4R in contrast to H3R.

TABLE 2
Ki (nM)

| Compound | H3R | H4R | H1R | H2R |
|---|---|---|---|---|
| Example 1 | 5.1 | ≧20,000 | 648 | 813 |
| Example 2 | 0.85 | ≧20,000 | 1764 | 894 |

Non-imidazole containing histamine H3 receptor antagonists disclosed in the literature generally have very poor pharmacokinetic properties (see J. Apelt, et al, J. Med. Chem. 2002, 45, 1128-1141). Compounds of this invention have markedly and unexpectedly improved pharmacokinetic properties. Male Sprague Dawley Rats (n=3 per dose arm) were separately dosed with 3 mg/kg iv or 10 mg/kg po of compound examples 8 and 19 (vehicle: water; dose volume: 1 mL/kg iv, 10 ml/kg po). Approximately 0.5 mL of blood was collected in heparin collection tubes at multiple time points over an 8 or 24 hour period for examples 8 and 19 respectively, and the samples were analyzed using LC/MS/MS. In this manner compound example 8 was found to have an oral bioavailability of 49% (AUC 0-8 hr; po/iv ratio) and an oral half-life of 12.2 hours. Compound example 19 was found to have an oral bioavailability of 100% (AUC 0-8 hr; poliv ratio) and an oral half-life of 12.4 hours.

From the above description, one skilled in the art can ascertain the essential characteristics of the present invention, and without departing from the spirit and scope thereof, can make various changes and modifications of the invention to adapt it to various usages and conditions. Thus, other embodiments are also within the claims.

1. A compound structurally represented by Formula I, 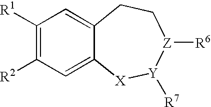
or pharmaceutically acceptable salts thereof, wherein:
R¹ and R² are independently H, or —OR³NR⁴R⁵, provided that one of R¹ and R² is —OR³NR⁴R⁵, and provided that only one of R¹ and R² is —OR³NR⁴R⁵;
R³ is (C₂-C₅)alkylene;
R⁴ is (C₁-C₄)alkyl;
R⁵ is (C₁-C₄)alkyl,
wherein R⁴ and R⁵ taken together with the nitrogen atom to which they are attached can form a piperidinyl or pyrrolidinyl ring;
X is CH₂ or CO;
Y and Z are —CH₂— or N, provided that one of Y or Z is N, and provided that only one of Y and Z is N;
R⁶ is hydrogen,
—(C₁-C₄)alkyl,
—CH₂-phenyl,
—CH₂ (C₃-C₇)cycloalkyl,
—CO₂R⁸,
—SO₂R⁹,
—CONHR¹⁰,
—COR¹¹,
—CH₂CH₂NR¹²R¹³, or
—CH₂R¹⁴;
R⁷ is hydrogen,
—(C₁-C₄)alkyl;
—CH₂-phenyl,
—CH₂(C₃-C₇)cycloalkyl,
—CO₂R⁸,
—SO₂R⁹,
—CONHR¹⁰,
—COR¹¹,
—CH₂CH₂NR¹²R¹³, or
—CH₂R¹⁴;
Wherein;
R⁸ is
—(C₁-C₄)alkyl, or
—(C₃-C₇)cycloalkyl;
R⁹ is
—(C₁-C₄)alkyl,
—(C₃-C₇)cycloalkyl, or
-phenyl;
R¹⁰ is
—(C₁-C₄)alkyl, or
—(C₃-C₇)cycloalkyl;
R¹¹ is
—(C₁-C₄)alkyl,
—(C₃-C₇)cycloalkyl,
—CH₂NR¹²R¹³, or
—(C₃-C₇)cycloalkyl, wherein optionally one or more of said carbons is replaced by N, NR¹⁰ or NCO₂R¹⁰;
R¹² is
-hydrogen, or
—(C₁-C₄)alkyl;
R¹³ is
-hydrogen,
—(C₁-C₄)alkyl,
—CO₂R¹⁰, or
-phenyl;
R¹⁴ is
—(C₁-C₄)alkyl, or
—(C₃-C₇)cycloalkyl, wherein optionally one or more of said carbons is replaced by N, NR¹⁰, or NCO₂R¹⁰.

2. The compound of claim 1, wherein R² is hydrogen and R¹ is —OR³NR⁴R⁵.

3. The compound of claim 2 wherein R³ is —CH₂CH₂CH₂—.

4. The compound of claim 3 wherein R⁴ and R⁵ taken together with the nitrogen atom to which they are attached form a piperidinyl ring.

5. The compound of claim 1 wherein Z is N and Y is CH₂.

6. The compound of claim 1 wherein Y is N and Z is CH₂.

7. The compound of claim 1 wherein X is CO.

8. The compound of claim 1 further represented by any one of formula (Example 1) to (Example 36) selected from the group consisting of:

| Example | |
|---|---|
| Number | Structure |
| 1 | 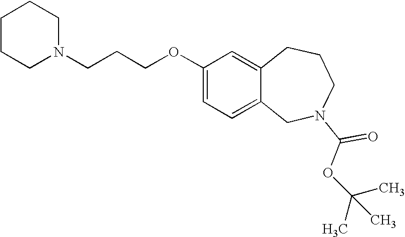 |
| 2 | 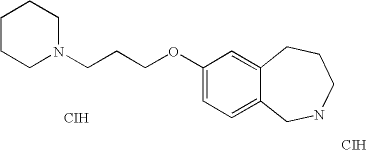 |
| 3 | 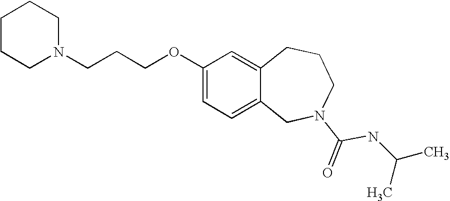 |
| 4 | 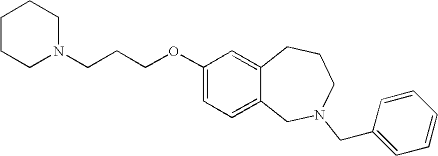 |
| 5 | 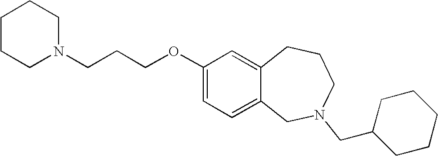 |
| 6 | 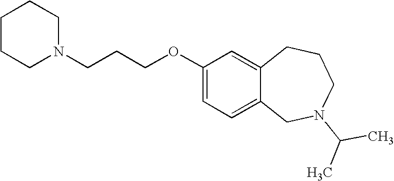 |
| 7 | 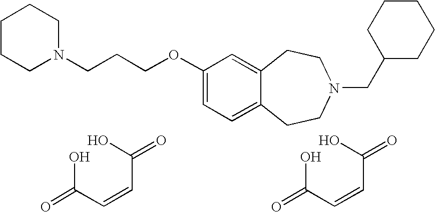 |
| 8 | 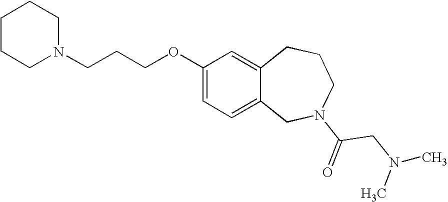 |
| 9 | 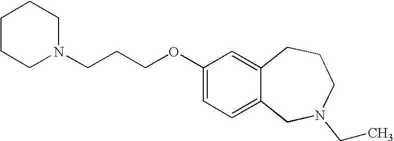 |
| 10 | 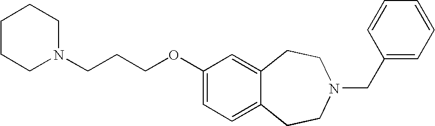 |
| 11 | 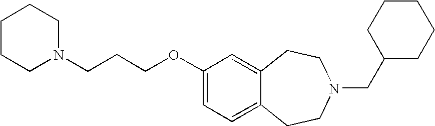 |
| 12 | 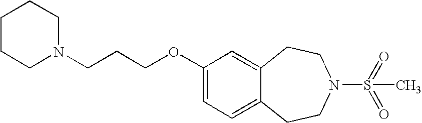 |
| 13 | 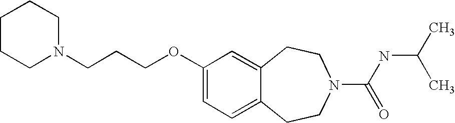 |
| 14 | 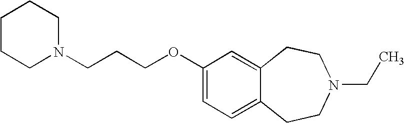 |
| 15 | 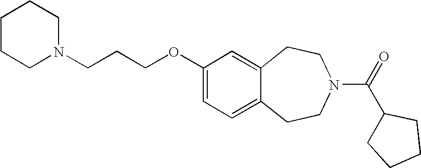 |
| 16 | 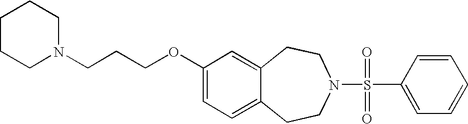 |
| 17 | 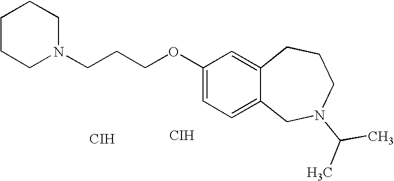 |
| 18 | 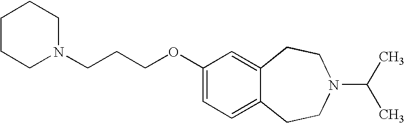 |
| 19 | 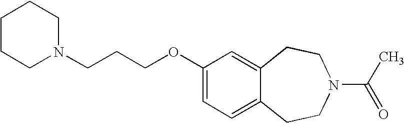 |
| 20 | 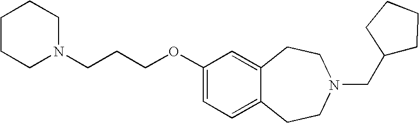 |
| 21 | 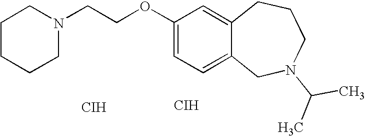 |
| 22 | 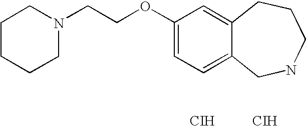 |
| 23 | 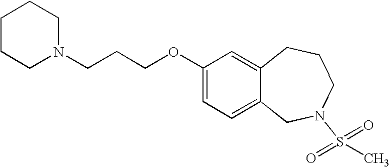 |
| 24 | 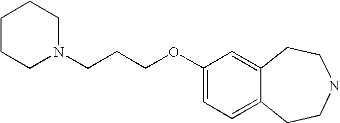 |
| 25 | 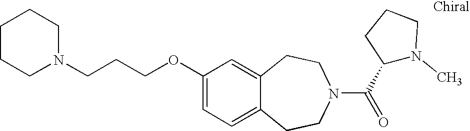 |
| 26 | 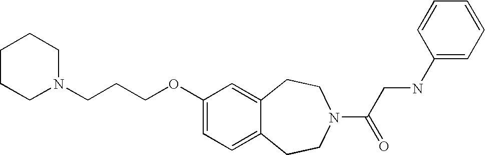 |
| 27 | 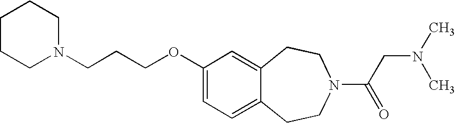 |
| 28 | 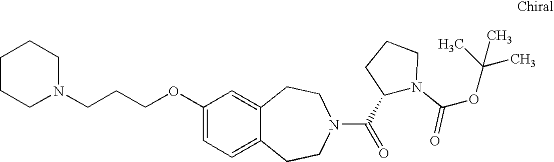 |
| 29 | 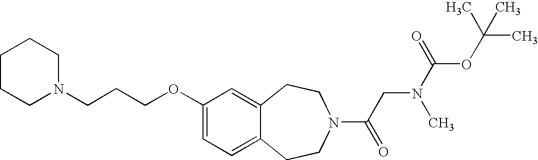 |
| 30 | 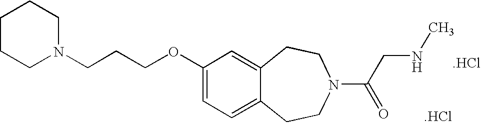 |
| 31 | 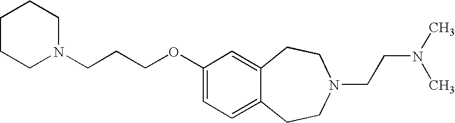 |
| 32 | 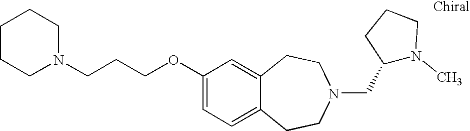 |
| 33 | 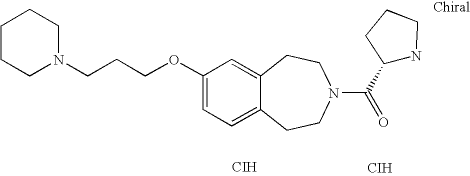 |
| 34 | 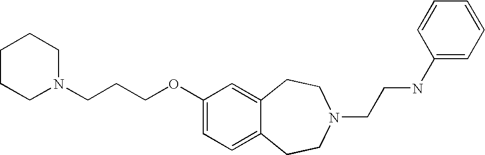 |
| 35 | 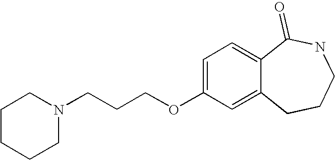 |
| 36 | 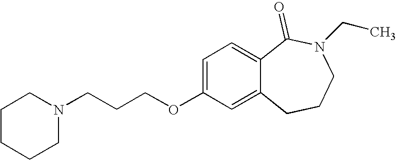 |

9. A pharmaceutical composition which comprises a compound of claim 1 or 8 and a pharmaceutically acceptable carrier.

10. (canceled)

11. (canceled)

12. (canceled)

13. A method for treatment or prevention of obesity which comprises administering to a subject in need of such treatment or prevention an effective amount of a compound of claim 1 or 8.

14. The method of claim 13 wherein the antagonist is a pharmaceutical composition of claim 9.

15. A method for treatment or prevention of a cognitive disorder which comprises administering to a subject in need of such treatment or prevention an effective amount of a compound of claim 1 or 8.

16. The method of claim 15 wherein the antagonist is a pharmaceutical composition of claim 9.
